(19) United States
(12) Patent Application Publication
(10) Pub. No.: US 2020/0316552 A1
(43) Pub. Date: Oct. 8, 2020

(54) MODULAR SYSTEMS FOR PERFORMING MULTISTEP CHEMICAL REACTIONS, AND METHODS OF USING SAME

(71) Applicant: SRI INTERNATIONAL, Menlo Park, CA (US)

(72) Inventors: Jin-Ping Lim, Menlo Park, CA (US); David Stout, Menlo Park, CA (US); Nathan Collins, Menlo Park, CA (US); Jason D. White, Menlo Park, CA (US); Jeremiah Malerich, Menlo Park, CA (US)

(21) Appl. No.: 16/496,834

(22) PCT Filed: Apr. 6, 2018
(86) PCT No.: PCT/US2018/026557

§ 371 Date: Sep. 23, 2019

Related U.S. Application Data
(60) Provisional application No. 62/482,515, filed on Apr. 6, 2017.

(51) Int. Cl. B01J 19/00; B01J 19/24; G16C 20/10; G01N 21/75; G01N 21/25

(52) U.S. Cl. CPC B01J 19/0006; B01J 19/24; G01N 21/25; G01N 21/75; G16C 20/10

(57) ABSTRACT

Disclosed are modular chemical reaction systems and methods of using such chemical reaction systems. The disclosed systems can have a substrate layer and a plurality of modules selectively mounted to an outer surface of the substrate layer. The substrate layer can include flow connectors that cooperate with the modules to form a fluid flow pathway for performing at least one step of a chemical reaction. At least one of the modules can be a process module, such as a reactor or separator. The modules can also include at least one regulator module. The system can also include at least one analysis device that analyzes at least one characteristic of the chemical reaction as the reaction occurs. The system can also include processing circuitry that monitors and/or optimizes the chemical reaction based on feedback received from the analysis device or other system components.

### CROSS-REFERENCE

This application claims priority to and the benefit of the filing date of co-pending U.S. Provisional Patent Application No. 62/482,515, filed Apr. 6, 2017. The entire disclosure of the aforementioned patent application is incorporated by reference herein.

### GOVERNMENT RIGHTS

This invention was made with government support under contract number W911N-16-0051 awarded by the United States Army. The government has certain rights in the invention.

### FIELD

Disclosed herein is a modular system for performing a chemical reaction, such as a multi-step chemical synthesis. More specifically, disclosed herein is a modular system for flow chemistry that allows for discovery, optimization, and low-volume production on the same platform. Also disclosed herein are methods for using such platforms.

### BACKGROUND

Traditional process development for the production of fine chemicals is a major undertaking, typically requiring several years of scale-up optimization and expert knowledge in a variety of fields. One of the major hurdles is that scale-up often begins at the bench scale in round bottom flasks, an inherently batch process. Unless an early switch to continuous flow chemistry is made early on, the successive generations of scale-up are direct increases in batch container size. However, the process conditions involved do not scale directly with the size, and re-optimization of the process is required at every step. Batch production is also less controllable than continuous production and needs rigorous quality control post-production to validate each batch.

In contrast, continuous production can be monitored directly, and can utilize immediate corrective feedback to keep the output within specification. However, no commercial system currently exists for continuous multi-step flow chemistry. Current systems allow for performance of single steps in flow, or perform them in a discontinuous fashion, i.e. using small volumes of reagents at a time. Additionally, these systems demonstrate little to no scalability for extending the number of steps.

The current state of the art in chemistry is restricted to the previously-mentioned flow chemistry systems, combinatorial screening systems, or traditional batch chemistry. Current flow chemistry systems are limited in the number of steps or amount of material they can process at a time and are also not designed to be expanded greatly. Typical chemistry to transform low-value starting materials into high-value products typically requires several discrete steps, sometimes in the dozens. Additionally, the other flow chemistry systems on the market require significant user intervention to reconfigure the system for a particular study, and must be manually re-connected to perform optimizations. In particular, existing systems do not allow for monitoring and changing reaction conditions and parameters of a reaction to optimize the reaction as the reaction occurs. Additionally, existing combination systems are incapable of performing reactions at elevated temperatures and pressures, and they cannot be used to produce large quantities of material in a continuous fashion. Finally, the traditional chemistry methods using round bottom flasks and conventional lab apparatus can produce a wide range of batch sizes, but continues to be restricted by batch operation, low pressures, and poor scalability, and requires significant time investment by highly-trained personnel.

Thus, there is a need for a robust modular platform for performing multi-step chemical analysis that allows scalability, provides easy re-configuration, reduces the overall cost, and is simple to operate. These and a number of additional features are provided by the following disclosure.

### SUMMARY

Disclosed herein, in exemplary aspects, is a modular chemical reaction system comprising a substrate layer, a plurality of modules, at least one analysis device, and processing circuitry. The substrate layer can have a substrate and a plurality of flow components positioned within the substrate. The substrate can have an outer surface. The plurality of modules can be selectively mounted to the outer surface of the substrate in overlying relation to the plurality of flow components. At least a portion of the plurality of modules can cooperate with at least a portion of the plurality of flow components to produce a first fluid flow pathway for performing at least one step of a first chemical reaction. The plurality of modules can comprise at least one monitoring module configured to produce at least one output indicative of at least one condition of the first chemical reaction. Each analysis device can be positioned in operative communication with the fluid flow pathway through at least one module of the plurality of modules and configured to produce at least one output indicative of at least one characteristic of the chemical reaction as the chemical reaction occurs. The processing circuitry can be communicatively coupled to the at least one monitoring module and the at least one analysis device. The processing circuitry can be configured to receive the outputs from the at least one monitoring module and the at least one analysis device to monitor the chemical reaction as the chemical reaction occurs. The plurality of modules and the flow components within the substrate layer can be configured for selective rearrangement within a minimal changeover period to produce a second fluid flow pathway for performing at least one step of a second chemical reaction, with the second fluid flow pathway being different than the first fluid flow pathway.

Also disclosed herein, in exemplary aspects, is a modular chemical reaction system having a substrate layer, a plurality of modules, at least one analysis device, and processing circuitry. The substrate layer can have a substrate and a plurality of flow components positioned within the substrate. The substrate can have an outer surface. The plurality of modules can be selectively mounted to the outer surface of the substrate in overlying relation to the plurality of flow components, and the plurality of modules can cooperate with the plurality of flow components to produce a first configuration that forms a first fluid flow pathway for performing at least one step of a first chemical reaction. The plurality of modules can comprise at least one monitoring module configured to produce at least one output indicative of at least one condition of the first chemical reaction. The at least one analysis device can be positioned in operative communication with the fluid flow pathway through at least one module of the plurality of modules and configured to produce at least one output indicative of at least one characteristic of the chemical reaction as the chemical reaction occurs. The processing circuitry can be communicatively coupled to the at least one monitoring module and the at least one analysis device. The processing circuitry can be configured to receive the outputs from the at least one monitoring module and the at least one analysis device to monitor the chemical reaction as the chemical reaction occurs. In use, the plurality of modules and the flow components within the substrate layer can be configured for selective rearrangement to a second configuration within a minimal changeover period to produce a second fluid flow pathway for performing at least one step of a second chemical reaction.

Also disclosed herein is a modular chemical reaction system having a substrate layer, a plurality of modules, at least one analysis device, and processing circuitry. The substrate layer can have a substrate and a plurality of flow components positioned within the substrate. The substrate can have an outer surface. The plurality of modules can be selectively mounted to the outer surface of the substrate in overlying relation to the plurality of flow components. The plurality of modules can cooperate with the plurality of flow components to form a fluid flow pathway for performing at least one step of a chemical reaction. The plurality of modules can include at least one process module and at least one regulator module. Each process module of the plurality of process modules can correspond to a location of a step of the chemical reaction. Each regulator module of the plurality of regulator modules can be positioned in fluid or thermal communication with the fluid flow pathway and configured to achieve, maintain, and/or measure one or more desired conditions of the chemical reaction. Each analysis device can be positioned in operative communication with the fluid flow pathway through at least one module and configured to produce at least one output indicative of at least one characteristic of the chemical reaction as the chemical reaction occurs. The processing circuitry can be communicatively coupled to the plurality of modules and the at least one analysis device. The processing circuitry can be configured to receive the at least one output from the at least one analysis device and to use the at least one output to adjust operation of the at least one process module and the at least one regulator module to optimize the chemical reaction. Optionally, the at least one process module can include a reactor or a separator.

Also disclosed herein is a modular chemical reaction system having a substrate layer, a surface-mount layer, and a plurality of sealing elements. The substrate layer can have a substrate and a plurality of flow components positioned within the substrate. The substrate can have an outer surface. The surface-mount layer can have a plurality of flow modules selectively mounted to the outer surface of the substrate in overlying relation to the plurality of flow components. Each flow module of the plurality of flow modules can be positioned in fluid communication with at least one flow component of the plurality of flow components at a respective interface. The plurality of sealing elements can be configured to establish a fluid-tight seal at each interface between a flow module of the plurality of flow modules and a flow component of the plurality of flow components. The plurality of flow modules and the plurality of flow components can cooperate to establish a fluid flow pathway for performing at least one step of a chemical reaction. At least one flow module of the plurality of flow modules can be a reactor or a separator.

A method of using the disclosed system can include introducing at least one reagent into the fluid flow pathway of the system. The method can further include performing a chemical reaction or sequence of chemical reactions using the at least one reagent. Optionally, the method can further include modifying the fluid flow pathway and running a second chemical reaction or sequence of chemical reactions using the modified fluid flow pathway.

Additional embodiments of the invention will be set forth, in part, in the detailed description, figures, and claims which follow, and in part will be derived from the detailed description, or can be learned by practice of the invention. It is to be understood that both the foregoing general description and the following detailed description are exemplary and explanatory only and are not restrictive of the invention as disclosed.

### BRIEF DESCRIPTION OF THE FIGURES

FIG. 1 is a schematic diagram depicting the relationship of the modular reaction systems and methods disclosed herein to an overall process for designing, performing, analyzing, and modifying chemical reactions.

FIG. 2A is a schematic diagram of a portion of an exemplary reaction system having a plurality of modules surface-mounted to a substrate layer as disclosed herein. FIGS. 2B-2C are schematic diagrams of a portion of an exemplary reaction system having a manifold layer, with FIG. 2B showing a side view of the system and FIG. 2C showing an end view of the system. FIG. 2D is a perspective view depicting the interaction between exemplary surface-mount components, flow connectors, and substrate and manifold layers as disclosed herein.

FIG. 3A is a schematic diagram providing a top view of an exemplary reaction system having surface-mounted process modules (reactors, separators), regulator modules (temperature modules, valves, pressure sensor modules), and analysis modules (for connection to an analysis device) as disclosed herein. As shown, the surface-mounted modules can be in communication with processing circuitry, such as, for example, a control module. FIG. 3B is a schematic view of an exemplary temperature module having a temperature sensor and a heating/cooling element. FIG. 3C is a schematic diagram depicting communication between a computing device and various components of a modular reactor system as disclosed herein.

FIG. 4 shows a schematic of an arrangement of exemplary components of a modular chemical reaction system as disclosed herein.

FIG. 5 shows exemplary dimensional requirements for the system components, which can comply with ANSI/ISA 76.00.02 standards.

FIG. 6 shows an exemplary flow path for one aspect of automated reconfiguration.

FIG. 7 shows an exemplary holdup tank module as disclosed herein.

FIG. 8 shows a photograph of various rector bases, including ⅛″ compression fittings and ¼″-28 flat bottom fittings.

FIG. 9 shows a photograph of an exemplary clamshell insulator (left) and an exemplary reactor assembly (right).

FIG. 10 shows a photograph of a 5 mL PFA reactor, a 10 mL-316SS reactor, and a 2 mL Hastelloy reactor.

FIG. 11 shows a photograph (a) and a schematic of an exemplary packed bed reactor (b).

FIG. 12 shows side, bottom, and perspective views of an exemplary gravity-based liquid-liquid separator module as disclosed herein.

FIG. 13 shows a photograph (a) and a schematic (b) of a UV/VIS and NIR flow cell module as disclosed herein.

FIG. 14 shows a photograph of an exemplary Raman flow cell module as disclosed herein.

FIG. 15 shows schematic views of an exemplary Raman flow cell as disclosed herein.

FIG. 16 shows results of an exemplary 2-step process as measured by DART-MS analysis as disclosed herein.

FIG. 17 shows results of an exemplary chlorination process as measured by FTIR as disclosed herein.

FIG. 18 shows a schematic of an exemplary 3-Step process as disclosed herein.

FIG. 19 shows a schematic of synthesis in batch.

FIG. 20 shows a schematic and results of one step synthesis using a commercially available system: (a) Friedel-Crafts reaction; (b)—alkylation reaction; (c)—epoxidation/opening reaction.

FIG. 21 shows a schematic of three-step synthesis of fluconazole on an exemplary reaction system as disclosed herein.

FIG. 22 shows a photograph of overall view of an exemplary modular reaction platform as disclosed herein.

FIG. 23 shows a photograph of a ventilated polycarbonate enclosure for an exemplary reaction platform as disclosed herein.

FIG. 24 shows a schematic of mapping synthetic routes on the baseline system configuration as disclosed herein.

FIG. 25A shows an photograph of an exemplary, non-limiting baseline platform configuration including specific surface mount components as disclosed herein. FIG. 25B is an exemplary schematic of the baseline configuration of FIG. 25A.

FIG. 26 shows an exemplary schematic of integrated user interface.

FIG. 27 shows an exemplary pathway for synthesis of diphenhydramine.

FIG. 28 shows an exemplary pathway for synthesis of fluconazole.

FIG. 29 shows an exemplary pathway for synthesis of tranexamic acid.

FIG. 30 shows an exemplary pathway for synthesis of hydroxychloroquine.

FIG. 31 shows an exemplary pathway for synthesis of diazepam.

FIG. 32 shows an exemplary pathway for synthesis of (S)-warfarin.

FIG. 33 shows an exemplary diagram showing ion counts as a function of time when the synthesis is switched from Diazepam to Warfarin in 1.2 hours. This limited time window can include time spent flushing the system, reconfiguring valve modules and other modules as appropriate, and starting the new reagents.

### DETAILED DESCRIPTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

### Definitions

As used herein, the singular forms “a,” “an” and “the” include plural referents unless the context clearly dictates otherwise. Thus, for example, reference to a “surface” includes aspects having two or more such surfaces unless the context clearly indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

As used herein, the term “changeover period” refers to the duration of a change between two configurations of a fluid flow pathway as disclosed herein. Optionally, the “changeover period” can refer to the duration of time associated with purging a first flow pathway (multiple times if necessary), changing valve positions within the disclosed system to produce a second flow pathway (before or after purging), and preparing the second flow pathway for initiating a second reaction or sequence of reactions. Additionally, or alternatively, the “changeover period” can include time during which surface-mount components and/or flow components of the disclosed modular reaction system are removed, replaced, added, or re-positioned as disclosed herein to produce the second fluid flow pathway. Although it is understood that the changeover period can vary depending upon the complexity of fluid flow pathways and specific reaction characteristics, it is contemplated that the disclosed system can provide a “minimal changeover period” compared to conventional systems. In exemplary aspects, a “minimal changeover period” can range from about 30 minutes to about 4 hours or, more typically, from about 1 hour to about 2 hours.

As used herein, the term “communicatively coupled” refers to any relationship between components that allows for transfer of information between the components as disclosed herein. Such relationships can include both wireless connections and direct electrical connections as are well known in the art.

References in the specification and concluding claims to parts by weight of a particular element or component in a composition or article, denotes the weight relationship between the element or component and any other elements or components in the composition or article for which a part by weight is expressed. Thus, in a composition or a selected portion of a composition containing 2 parts by weight of component X and 5 parts by weight component Y, X and Y are present at a weight ratio of 2:5, and are present in such ratio regardless of whether additional components are contained in the composition.

A weight percent of a component, unless specifically stated to the contrary, is based on the total weight of the formulation or composition in which the component is included.

It is understood that if a machine-readable medium is described herein, it may include any mechanism for storing or transmitting information in a form readable by a machine. For example, a machine-readable medium may include any suitable form of volatile or non-volatile memory. Modules, data structures, function blocks, and the like are referred to as such for ease of discussion, and are not intended to imply that any specific implementation details are required. For example, any of the described modules may be combined or divided into sub-modules, sub-processes or other units as may be required by a particular design or implementation. In the drawings, specific arrangements or orderings of schematic elements may be shown for ease of description. However, the specific ordering or arrangement of such elements is not meant to imply that a particular order or sequence of processing, or separation of processes, is required in all embodiments. In general, schematic elements used to represent instruction blocks or modules may be implemented using any suitable form of machine-readable instruction, and each such instruction may be implemented using any suitable programming language, library, application programming interface (API), and/or other computer programming mechanisms.

Similarly, schematic elements used to represent data or information may be implemented using any suitable electronic arrangement or data structure. Further, some connections, relationships or associations between elements may be simplified or not shown in the drawings so as not to obscure the disclosure. This disclosure is to be considered as exemplary and not restrictive in character, and all changes and modifications that come within the spirit of the disclosure are desired to be protected.

The present invention may be understood more readily by reference to the following detailed description of preferred embodiments of the invention and the examples included therein and to the Figures and their previous and following description.

### INTRODUCTION

Disclosed herein are modular chemical reaction systems. It is contemplated that the disclosed systems can comprise components that simulate the piping and reactors of a conventional chemical plant at the bench scale, allowing for direct scale-up from laboratory to production. Additionally, the disclosed system can be integrated with a variety of analytical technologies and sensors, which populate a database to create feedback control models for maintaining quality. The system also allows for processing of hazardous reagents and temperatures and pressures unachievable by conventional batch experimentation, while also allowing for automation to perform complete designs of experiments (DoEs) to further increase the productivity of the experimenter. These and other aspects of the disclosed system can enable the system to accelerate process development and even completely change how translational chemistry is performed.

As further described herein, the disclosed system is a robust, modular system for flow chemistry that can allow for discovery, optimization, and low-volume production on the same platform. In exemplary applications, miniaturized valving automation modules can be used to create a general synthesizer that can emulate a wide variety of chemical engineering unit operations and reactor sizes. This capability can allow the performance of an incredibly broad range of chemistry schemes without having to manually reconfigure the system. When connected to the appropriate instrumentation and controls, this can create a fully-adaptive, reprogrammable chemical synthesis array. Additionally, system performance characteristics can be characterized to allow scale-up, allowing the learnings at one scale to be applied directly to an industrial-scale production platform without intermediate scale-up and re-optimization.

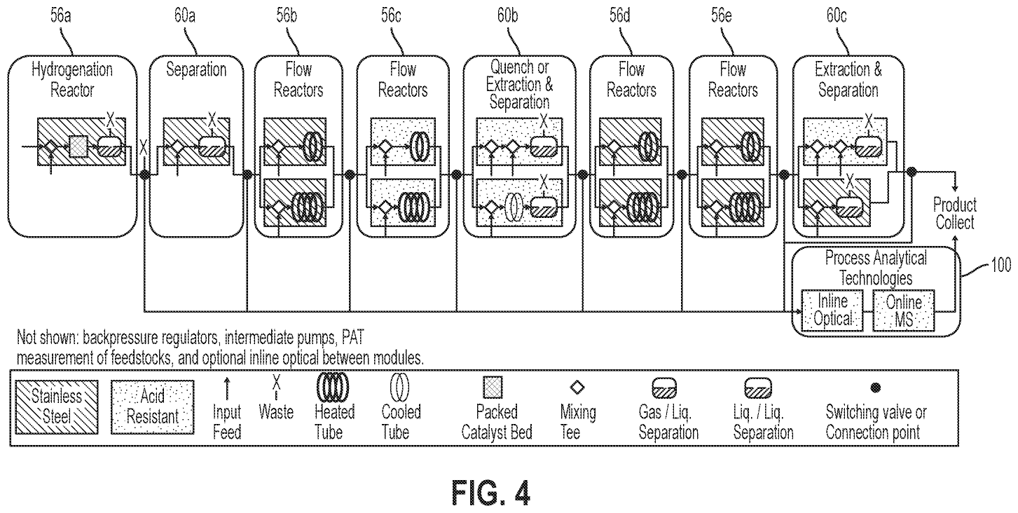

As further described herein, the disclosed system can include a variety of fully-modular surface-mount components that replicate conventional plant operations at a miniaturized scale. Optionally, in exemplary aspects, the system can comply with ANSI/ISA 76.00.02-2002 and IEC 62339-1:2006 standards for gas sampling analysis as depicted in FIG. 4. The disclosed system can include new surface-mount components, which span multiple positions on the supporting substrate channels. Also disclosed herein are several types of reactors including heated and cooled residence time reactors, mixers, separators, and storage tanks. These improved surface-mount components can have similar flow characteristics and operating performances, thereby allowing for plug-and-play reorganization of the modules as needed.

The system can also use custom connectors and manifolds to maintain liquid flow characteristics throughout the system. Disclosed herein are new flow components having a reduced flow cross-section that is matched to the reaction tubing and generates superior plug flow characteristics compared to existing designs. These flow components can comprise chemically-resistant materials and can be made at low cost and using conventional orbital welding techniques. Additionally, disclosed herein are bypass flow components that allow new interconnects to be made in the field for custom lengths, allowing for direct connection of two non-adjacent surface-mount components. The disclosed bypass connectors can greatly reduce the cost of long connections, reduce the tortuosity of the flow path, and allow for in-line monitoring of flow.

In contrast to commercially available fluid distribution components, which are designed for process analyzer and sample-handling systems, the disclosed system can perform chemical reactions as well as purification steps directly within the constraints of the described platform. Additionally, the design of the modules can allow for expansion of the system by connecting additional modules to the system, while using the same support architecture.

As further described herein, the disclosed system can include modular reactors, which can assemble a single reaction “block” by putting together several modules and connectors. This approach is radically different from the fully-integrated modules that are known in the art. Beyond basic heated/cooled residence time modules, the disclosed system can include modules that allow for mixing, catalysis, separation, and the like. Additionally, the disclosed modules can be automated in such a way that allows for computer-controlled optimization instead of manual reconfiguration. Each of these modules can be individually-addressable, further enhancing the flexibility of the system. The system can also be designed for end-to-end continuous use, which further enhances the similarities between the lab-scale and production-scale reactors, allowing for pinpointing and troubleshooting of problems much earlier in the timeline, and allowing for direct transition to production mode after optimizing on the lab-scale system.

It is contemplated that the disclosed system has the potentially to completely revolutionize how exploratory chemistry is performed. Currently, the paradigm for drug discovery is discovery at the bench-scale by experienced chemists. This is typically followed by successive scale-ups of increasingly larger batches, which must be re-optimized at every scale-up stepdue to wildly different reactor characteristics, such as heat and mass transfer. The drug discovery process is also extremely time-consuming, with some reaction schemes requiring dozens of processing steps, which could span weeks, with no guarantee of success. It is contemplated that the disclosed automated optimization approach can allow compression of this development phase into hours or days allowing for testing of hundreds of reaction schemes rather than one or two. It is contemplated that such a fully-automated system can be run 24/7 (i.e., at all times, in a continuous fashion) with little to no human intervention, all while maintaining high degrees of consistency and safety.

Example implementations of the disclosed system include a generalized chemical synthesizer configured for high-value fine chemicals businesses, such as small molecule pharmaceuticals. Another aspect of the disclosed system provides automated chemical optimization techniques, which can use deep learning strategies on accumulated process data, which can be stored in a central knowledge repository and analyzed to benefit all users of the integrated system.

### Modular Chemical Reaction Systems

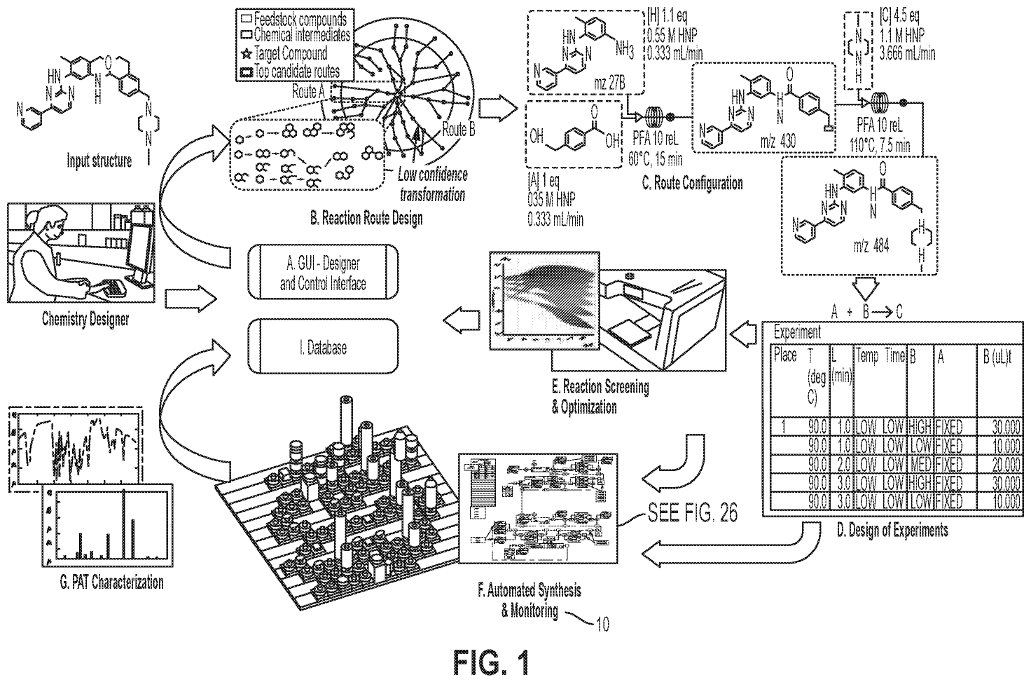

Disclosed herein, in various aspects and with reference to FIGS. 1-3C, is a modular chemical reaction system 10. The system 10 can have a substrate layer 20 and a surface-mount layer 40 including a plurality of modules 50 as further disclosed herein. The system 10 can further comprise a plurality of sealing elements 90.

In use, and as schematically depicted in FIG. 1, it is contemplated that the modular chemical reaction system 10 can provide automated chemical synthesis and monitoring capabilities that can be incorporated into a comprehensive system for designing, simulating, screening, performing, analyzing, and modifying/optimizing chemical reactions. As further disclosed herein, it is contemplated that the disclosed system 10 can provide modularity that permits rapid reconfiguration (optionally, rearrangement) of system components to quickly change fluid flow pathways associated with multiple, varying reactions. In some aspects, reconfiguration means selecting alternative pathways within the system having defined pathways and pre-positioned modules and/or analysis devices. In these aspects, it is contemplated that the defined pathways can be separated by valve modules as disclosed herein, which can be adjusted to modify the flow of fluid within and among the defined pathways. In other aspects, reconfiguration can include physically adding new modules or analysis devices to the disclosed system 10. Additionally, or alternatively, reconfiguration can include removing or replacing at least one module or analysis device as disclosed herein. It is further contemplated that the disclosed system 10 can provide a framework for performing multiple chemical reactions using a single configuration of reaction modules. Still further, it is contemplated that the disclosed system 10 can provide monitoring capabilities during the performance of a chemical reaction that have previously been unachievable. Still further, it is contemplated that the disclosed system 10 can control and/or optimize reaction conditions based on feedback received from various modules and analysis devices as a reaction occurs.

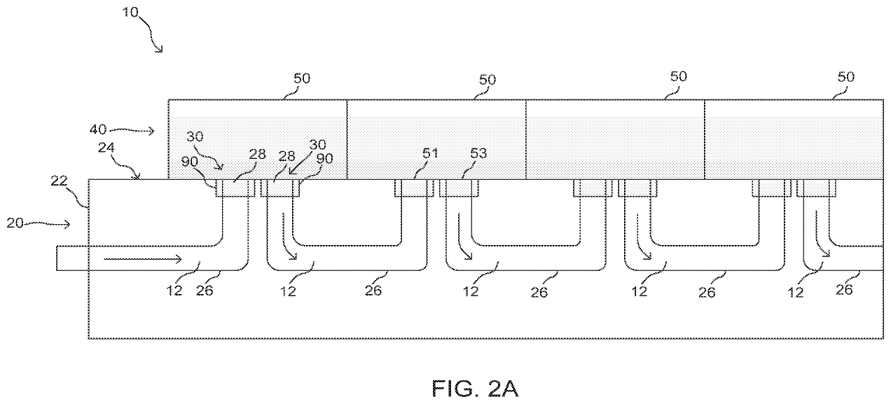

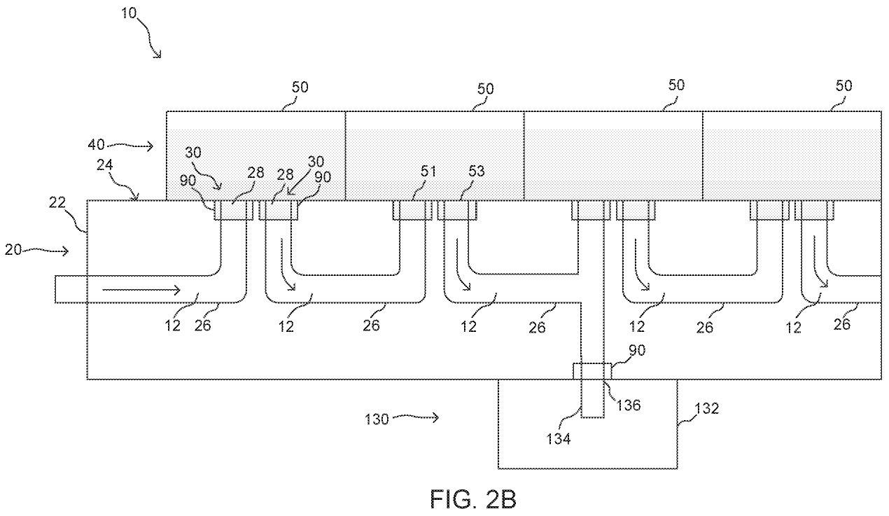

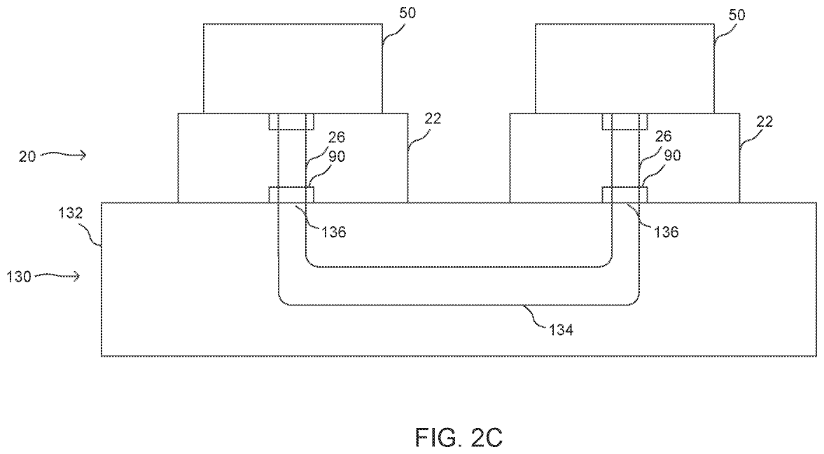

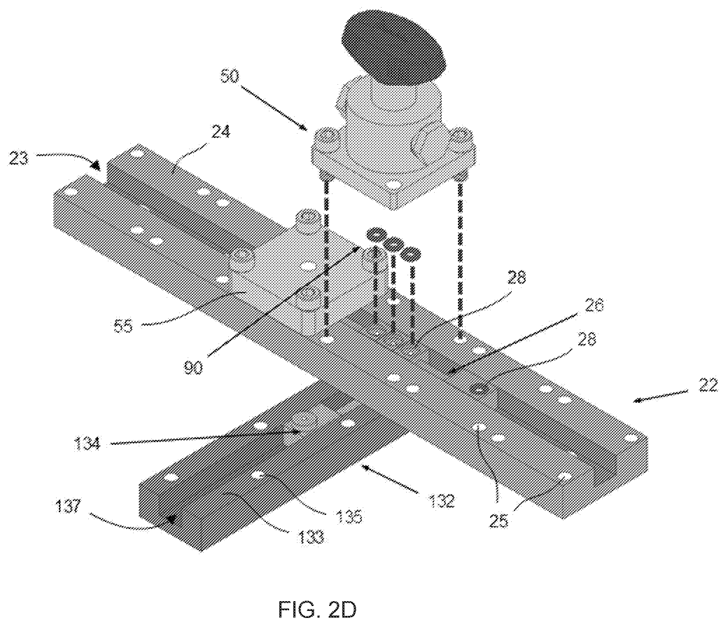

In exemplary aspects, and with reference to FIGS. 2A-2D, the substrate layer 20 can have a substrate 22 and a plurality of flow components (e.g., flow connectors 26) positioned within the substrate. In these aspects, the substrate 22 can have an outer surface 24. Optionally, in exemplary aspects, the substrate 22 can comprise a plurality of substrate bodies that are selectively positioned in parallel to establish a framework for parallel fluid passageways as disclosed herein. Although the substrate bodies are generally described herein as being in parallel, it is contemplated that the substrate bodies can be positioned in any desired configuration, including perpendicular and angled configurations. Alternatively, it is contemplated that the substrate 22 can be a single contiguous platform structure. In exemplary aspects, the substrate layer 20 (and the manifold layer disclosed further herein) can be configured for selective attachment to an underlying grid support structure defining a plurality of openings for receipt of fasteners to secure the substrate layer and/or manifold layer to the grid support structure.

Optionally, each module 50 of the plurality of modules can have at least a first inlet 51 and a first outlet 53 as depicted in FIG. 2A. However, it is contemplated that some modules can be configured for storage of material and/or otherwise only include an inlet 51 or an outlet 53.

In additional aspects, the plurality of modules 50 of the surface-mount layer 40 can be selectively mounted to the outer surface 24 (e.g., upper surface) of the substrate 22 in overlying relation to the plurality of flow components (e.g., flow connectors 26). In these aspects, it is contemplated that the plurality of modules 50 can include a plurality of flow modules 52 that receive fluid that forms a portion of a fluid flow pathway within the system 10. It is further contemplated that each flow module 52 of the plurality of flow modules can be positioned in fluid communication with at least one flow component (e.g., flow connector 26) of the plurality of flow components at a respective interface 30 as shown in FIG. 2A. In further aspects, the plurality of sealing elements 90 can be configured to establish a fluid-tight seal at each interface 30 between a flow module 52 of the plurality of flow modules and a flow component (e.g., flow connector 26) of the plurality of flow components. As further disclosed herein, at least a portion of the plurality of flow modules 52 and at least a portion of the plurality of flow components (e.g., flow connectors 26) can cooperate to establish a fluid flow pathway 12 (e.g., a first fluid flow pathway) for performing at least one step of a chemical reaction or series of chemical reactions. As further disclosed herein, it is contemplated that the configuration of the flow modules and flow components can be selectively modified to produce a second fluid flow pathway that differs from the first fluid flow pathway. Optionally, in exemplary aspects, the fluid flow pathway can be a liquid flow pathway. In these aspects, it is contemplated that the sealing elements 90 can be configured to establish liquid-tight seals at each interface 30 between a flow module 52 and a flow connector 26. In further exemplary aspects, it is contemplated that the chemical reaction can be a continuous flow, multi-step chemical reaction.

In additional aspects, each flow connector 26 can be configured to selectively form a portion of the fluid flow pathway 12 for performing at least one step of the chemical reaction. Alternatively, each flow connector 26 can be configured to selectively be disengaged from flow connectors forming the fluid flow pathway such that the flow connector is not in fluid communication with the fluid flow pathway. In exemplary aspects, each flow connector 26 can have opposing inlet/outlet openings 28 that can function as an inlet or an outlet depending upon the direction of fluid flow in a particular flow pathway configuration. As depicted in FIG. 2D, it is contemplated that the flow connectors 26 can be positioned within a channel 23 extending along the length of the substrate 22. In further aspects, it is contemplated that the outer surface 24 of the substrate 22 can define connection openings 25 that are configured to permit fastening of a surface-mounted component (e.g., module) to the substrate. It is further contemplated that the inlet/outlet openings 28 of the flow connectors 26 can project upwardly or downwardly from adjoining portions of the flow connector to engage the inlets or outlets of modules or other flow connectors as disclosed herein.

In exemplary aspects, it is contemplated that each module 50 of the plurality of modules can have a common base structure that includes a plurality of openings that are configured to receive fasteners (e.g., bolts or screws) for mounting the module to the outer surface 24 of the substrate 22. In these aspects, it is contemplated that the locations of the openings within the base structure of each module 50 can be complementary to corresponding connection openings 25 defined within the substrate layer 20. It is further contemplated that the common base structure can include a common dimensional profile, such as, for example and without limitation, a square profile, which can optionally include length and width dimensions of about 1.5 inches. In some exemplary aspects, the disclosed modules 50 can be directly mounted to a substrate 22 as disclosed herein. Alternatively, in other exemplary aspects, and as shown in FIG. 2D, the disclosed modules 50 can be mounted to a base plate 55 that is in turn mounted to a substrate 22 as disclosed herein.

Optionally, in further aspects, and as shown in FIGS. 2B-2D, the modular chemical reaction system 10 can further comprise a manifold layer 130. In these aspects, the manifold layer 130 can comprise at least one manifold body 132 underlying the substrate layer 20. Optionally, the manifold body 132 can comprise a plurality of manifold bodies that are selectively positioned in parallel to establish a framework for parallel fluid passageways as disclosed herein. Alternatively, it is contemplated that the manifold body 132 can be a single contiguous platform structure. In use, it is contemplated that the manifold bodies 132 can be oriented perpendicular to the substrates 22 disclosed herein in order to provide for conveyance of reaction components among parallel substrates. Alternatively, in other aspects, a manifold body 132 can be oriented parallel to (or directly underlie) a substrate body to permit bypassing of certain reaction modules aligned with a particular substrate body. In exemplary aspects, it is contemplated that the plurality of flow connectors 26 of the system can comprise a first plurality of flow connectors 26 positioned within the substrate layer 20 and a second plurality of flow connectors 134 positioned within the manifold layer 130. Each flow connector 134 of the manifold layer 130 can have opposing inlet/outlet openings 136 that can function as an inlet or an outlet depending upon the direction of fluid flow in a particular flow pathway configuration. As depicted in FIG. 2D, it is contemplated that the flow connectors 134 can be positioned within a channel 137 extending along the length of the manifold body 132. In further aspects, it is contemplated that the manifold body 132 can have an outer surface 133 that defines connection openings 135 that are configured to permit fastening of a substrate 22 to the manifold body. It is further contemplated that the inlet/outlet openings 136 of the flow connectors 134 can project upwardly or downwardly from adjoining portions of the flow connector to engage the inlets or outlets of modules or other flow connectors as disclosed herein.

It is contemplated that the disclosed flow connectors 26, 134 of the substrate layer and the manifold layer can be provided in a range of varying lengths and shapes to permit connection with other flow connectors and a variety of modules as disclosed herein.

Although depicted in FIGS. 2B-2D as having two layers (the substrate layer 20 and the manifold layer 130) beneath the surface-mount layer 40, it is contemplated that the disclosed system can have additional layers below the manifold layer 130 to permit further fluid pathway modification.

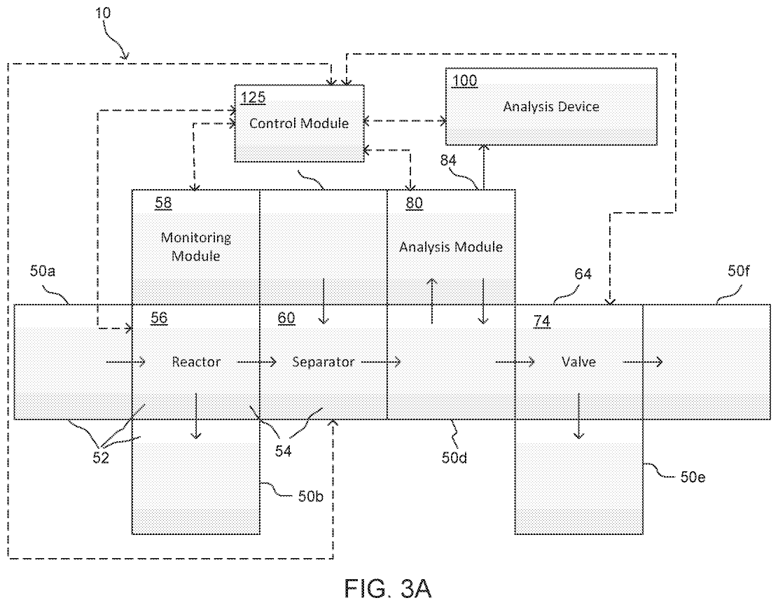

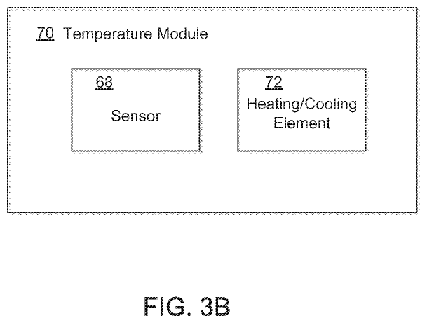

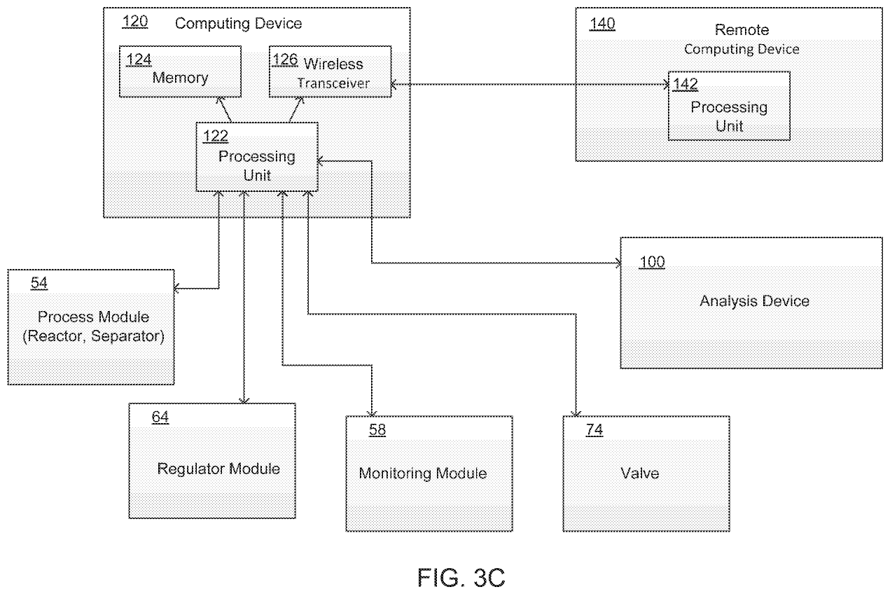

In additional aspects, and with reference to FIGS. 3A-3B, the plurality of modules 50 can comprise at least one monitoring module 58 that is configured to produce at least one output indicative of at least one condition of a chemical reaction. In these aspects, it is contemplated that the at least one monitoring module 58 (optionally, a plurality of monitoring modules) can be communicatively coupled to processing circuitry as further disclosed herein. Exemplary conditions that can be monitored by the at least one monitoring module 58 include, but are not limited to temperature, pressure, flow rate, an identification of products generated by a reaction, a rate of consumption of a reagent, an identification of side products, yield, selectivity, purity, and the like. It is contemplated that the at least one monitoring module can comprise sufficient sensors, hardware, or processing components that are capable of generating outputs corresponding to the conditions monitored by the at least one monitoring module 58.

In further exemplary aspects, at least one flow module 52 of the plurality of flow modules can be a process module 54 that can correspond to a location of a step of the chemical reaction. Optionally, each process module 54 disclosed herein can also serve as a monitoring module 58, where the process module 54 is also configured to provide at least one output to processing circuitry as further disclosed herein. Examples of such process modules 54 include a reactor 56 or a separator 60 as further disclosed herein. In one aspect, when the at least one process module 52 comprises a reactor 56, it is contemplated that the reactor can be a heated tube reactor, a packed-bed reactor, or combinations thereof. However, it is contemplated that other reactors can be used, provided they have the surface-mount capabilities disclosed herein. In another aspect, when the at least one process module 52 comprises a separator 60, the separator can be a liquid/liquid separator or a gas/liquid separator. In one optional aspect, the separator 60 can comprise a membrane-based liquid-liquid separator as further disclosed in the Examples section of this application. In another optional aspect, the separator 60 can comprise a gravity-based liquid-liquid separator as further disclosed in the Examples section of this application. In this aspect, and as further described herein, it is contemplated that the gravity-based liquid-liquid separator can be configured for use under under pressures above atmospheric conditions as is conventional. It is further contemplated that the disclosed gravity-based liquid-liquid separator can comprise glass that permits visibility of the separation process. It is still further contemplated that the disclosed gravity-based liquid-liquid separator can provide inlet and outlet flow paths that travel in a common plane rather than in different planes as is conventional. In further aspects, it is contemplated that the separator 60 can comprise a gravity-based gas-liquid separator as further disclosed in the Examples section of this application.

Optionally, in exemplary configurations, the plurality of flow modules 52 of the system can comprise at least one reactor 56 and at least one separator 60.

Optionally, in exemplary aspects, it is contemplated that each flow connector 26 of the substrate layer 20 (and each flow connector 134 of the manifold layer 130, when present) can have a consistent inner diameter along its entire length (optionally, ranging from about 0.04 inches to about 0.08 inches). Optionally, in these aspects, the at least one flow module 52 of the system 10 can comprise a reactor 56 and/or separator 60, and at least one of the fluid inlet 51 and the fluid outlet 53 of the at least one flow module 52 can share a consistent inner diameter with an adjacent flow connector 26 of the plurality of flow connectors. Optionally, in still further exemplary aspects, at least a portion of the flow connectors 26, 134 (optionally, each flow connector) of the plurality of flow connectors can comprise Hastelloy C276. In contrast to known flow connectors, which have a variable inner diameter at various locations, it is contemplated that the disclosed flow connectors can provide improved performance by minimizing dead space and providing improved fluid flow (particularly in liquid reactions).

Optionally, in further exemplary aspects, the plurality of modules 50 of the modular chemical reaction system 10 can comprise at least one regulator module 64. Optionally, in these aspects, each regulator module 64 disclosed herein can also serve as a monitoring module 58, where the regulator module 64 is also configured to provide at least one output to processing circuitry as further disclosed herein. In exemplary aspects, it is contemplated that each regulator module 64 can be positioned in fluid or thermal communication with the fluid flow pathway 12 and configured to achieve, maintain, and/or measure one or more desired conditions of the chemical reaction. Optionally, the plurality of modules 50 of the system 10 can include at least one process module 54 and at least one regulator module 64. Exemplary regulator modules 64 include, for example and without limitation: a check valve, a tee filter, a flow regulator, a pressure sensing module, a pressure relief valve, a back pressure regulator, a tube adaptor, a valve, a pump, a flow stream selector, a control valve module, a temperature monitoring module, a temperature control module, a heater, a cooler, or combinations thereof. In exemplary aspects, it is contemplated that at least one regulator module 64 can comprise a sensor (e.g., a temperature, pressure, or flow sensor) positioned in fluid and/or thermal communication with a portion of the fluid flow pathway and configured to produce an output indicative of at least one characteristic of fluid (e.g., liquid) within the regulator module (in this case, a flow module as well). For example, as shown in FIG. 3 B, a temperature module 70 can comprise a temperature sensor 71 and, optionally, also comprise heating and/or cooling element 72 as is known in the art and further disclosed herein. In other exemplary aspects, it is contemplated that at least one regulator module 64 can be configured to effect adjustment of at least one property of the fluid within the fluid flow pathway. For example, a valve module 74 can be configured to move among at least first and second positions to modify flow of fluid through the fluid flow pathway. Optionally, it is contemplated that each valve module 74 can comprise a servo motor and position sensors (e.g., encoders) that are communicatively coupled to the processing circuitry as further disclosed herein to permit selective monitoring and/or control of valve positioning.

In exemplary aspects, it is contemplated that the system 10 can comprise at least one analysis device 100. In these aspects, each analysis device 100 can be positioned in operative communication with the fluid flow pathway 12 through at least one module 50. As used in this context, the term “operative communication” can refer to any form of communication necessary to permit analysis by an analysis device 100 as disclosed herein. It is further contemplated that each analysis device 100 can be configured to produce at least one output indicative of at least one characteristic of the chemical reaction as the chemical reaction occurs. In further aspects, each analysis device 100 can comprise: a UV-Vis spectrometer, a near-infrared (NIR) spectrometer, a Raman spectrometer, a Fourier Transform-Infrared (FT-IR) spectrometer, a nuclear magnetic resonance (NMR) spectrometer, or a mass spectrometer (MS). More generally, it is contemplated that the analysis device 100 can be any conventional Process Analytical Technologies (PAT) device that is suitable for use in at least one step of a chemical reaction or a series of chemical reactions. It is further contemplated that one or more analysis device can be placed along the flow path of the system 10, wherein each of the analysis devices can send output analyses to the processing circuitry for monitoring or further optimizing the one step of the chemical reaction or the series of chemical reactions being performed. In exemplary aspects, the plurality of modules 50 can comprise at least one analysis module 80 having at least a second outlet 84 that is positioned in operative communication with an analysis device 100 as disclosed herein. Optionally, in these aspects, it is contemplated that the analysis module 80 can be positioned upstream of at least one other flow module of the plurality of flow modules. However, in other aspects, it is contemplated that the analysis module 80 can be positioned at a location corresponding to an end or completion of a reaction. In some exemplary aspects, it is contemplated that the analysis module 80 can be communicatively coupled to the analysis device 100. In these aspects, it is contemplated that the analysis module 80 can serve as a monitoring module 58 as further disclosed herein.

In further exemplary aspects, the system 10 can comprise processing circuitry 110. In these aspects, it is contemplated that the processing circuitry 110 can be communicatively coupled to at least one module of the plurality of modules 50 (e.g., at least one monitoring module 58) and the at least one analysis device 100. It is further contemplated that the processing circuitry 110 can be configured to receive the at least one output from the at least one module (e.g., monitoring module 58). Optionally, the processing circuitry 110 can receive a plurality of outputs from a plurality of modules (e.g., monitoring modules), either sequentially or simultaneously. Optionally, the processing circuitry 110 can use the at least one output to adjust operation of at least one module 50 (e.g., a process module 54 and/or a regulator module 64) to optimize the chemical reaction or a portion of the chemical reaction. Additionally, or alternatively, it is further contemplated that the processing circuitry 110 can be configured to receive the at least one output from the at least one analysis device 100. Optionally, the processing circuitry 110 can receive a plurality of outputs from a plurality of analysis devices, either sequentially or simultaneously. Optionally, the processing circuitry 110 can use the at least one output to adjust operation of at least one module 50 (e.g., a process module 54 and/or a regulator module 64) to optimize the chemical reaction or a portion of the chemical reaction. In exemplary aspects, the processing circuitry can simultaneously or sequentially receive outputs from at least one module (e.g., monitoring module) and at least one analysis device as a reaction occurs.

In additional aspects, it is contemplated that the processing circuitry can respond to the outputs received from the monitoring module 58 and/or the analysis device 100 to adjust specific reaction parameters based upon pre-set conditions saved within the processing circuitry (i.e., within a memory of the processing circuitry) or based upon adjustments made through user inputs (i.e., through user interfaces positioned in communication with the processing circuitry).

In some aspects, a user can manually trigger a change in any one of the modules by changing one or more parameters in the processing circuitry based upon outputs from one or more monitoring modules and/or one or more analysis devices as disclosed herein.

In some aspects, the disclosed processing circuitry (optionally, in the form of a controller) can be used to automatically orchestrate changes to one or more modules of the system based upon outputs from one or more monitoring modules and/or one or more analysis devices as disclosed herein, where changes are based upon a pre-set trigger (such as a pre-determined threshold temperature or yield parameter), which can optionally be stored in the memory of the processing circuitry. For example, if the temperature of a given reaction is beyond a pre-set threshold temperature, the processing circuitry can send instructions/commands to the corresponding temperature regulator to reduce the temperature for that reactor for that particular reaction until the temperature drops below the threshold temperature value.

An exemplary schematic flow diagram of a system 10 is provided in FIG. 3A. Each contiguous box corresponds to a respective module 50; although shown contiguously, it is understood that the modules need not be in direct contact with one another. The solid arrows within the contiguous boxes represent flow of fluid within a fluid pathway as disclosed herein, while the dashed arrows represent communication among system components. Module 50a receives an inlet feed of fluid, and an underlying flow connector delivers the fluid to the adjacent separator module 60. Separator module 60 is shown in thermal communication with monitoring module 58 and in fluid communication with reactor 56 and module 50b, each of which receives a different separation product. The monitoring module 58 can monitor one or more conditions during the separation step. Optionally, in one example, the monitoring module 58 can be a temperature module 70 that can be configured to monitor temperature during the separation step and optionally be configured to provide additional heat or cooling to maintain a desired or selected temperature as disclosed herein. Module 50c represents another inlet feed source that delivers additional fluid into reactor 56. The products of the reaction within reactor 56 are delivered to module 50d, which is in fluid communication with analysis module 80, which is in turn in operative communication with an analysis device 100 as disclosed herein. Module 50d is also in fluid communication with valve 74, which can be selectively adjusted to direct fluid toward either module 50e or module 50f. As further disclosed herein, it is contemplated that at least a portion of the disclosed modules can be communicatively coupled to the processing circuitry 110, which can be used to provide active feedback and/or modification to the surface-mounted system components.

FIG. 3C depicts an exemplary configuration in which the surface-mounted components of the system can be communicatively coupled to processing circuitry, such as a computing device 120 (optionally, a plurality of computing devices) as further disclosed herein. Non-limiting examples of the computing device 120 include a desktop computer, a laptop computer, a central server, a mainframe computer, a tablet, a smartphone, and the like. In exemplary aspects, the computing device 120 can be positioned in the vicinity of the system 10. For example, in various exemplary aspects, and as shown in FIG. 3A, it is contemplated that at least one computing device 120 of the system can be a control module 125, which can be selectively surface-mounted as disclosed herein or otherwise positioned in the vicinity of the surface-mounted components. In these aspects, it is contemplated that a plurality of control modules 125 can be selectively positioned within the system 10 to form desired feedback loops as disclosed herein.

As shown in FIG. 3C, it is contemplated that the computing device 120 can comprise a processing unit 122 (e.g., a CPU) that is in communication with a memory 124. In exemplary aspects, the processing unit 122 can be communicatively coupled to at least one module 50 of the system 10 using conventional wired (e.g., cable, USB) or wireless (WiFi, Bluetooth) communication protocols. Additionally, or alternatively, it is contemplated that the processing unit 122 can be communicatively coupled to at least one analysis device 100 using conventional wired (e.g., cable, USB) or wireless (WiFi, Bluetooth) communication protocols. It is contemplated that the processing unit 122 can be communicatively coupled to at least one monitoring module 58 (e.g., a plurality of monitoring modules) as further disclosed herein. In exemplary aspects, the processing unit 122 can be communicatively coupled to at least one process module 54. Additionally, or alternatively, in further exemplary aspects, the processing unit 122 can be communicatively coupled to at least one regulator module 64, such as a temperature module 70 or a valve 74.

Optionally, the computing device 120 can comprise a wireless transceiver 126 (e.g., a WiFi or Bluetooth radio) that is configured to wirelessly transmit and receive information. In exemplary aspects, it is contemplated that the wireless transceiver 126 can be communicatively coupled to a remote computing device 140, such as a tablet, a smartphone, or other computing device positioned at a location remote from the system. In these aspects, the remote computing device can be configured to provide remote user inputs or monitor progress of an ongoing reaction based upon outputs received from the computing device 120 (optionally, through WiFi, a cellular network, or a Cloud-based system).

FIG. 3A also includes an exemplary schematic communication diagram of the system 10. As shown, it is contemplated that a plurality modules of the system can be communicatively coupled to processing circuitry, shown here as a control module 125. During performance of at least one step of a reaction using the disclosed system, it is contemplated that one or more monitoring modules 58 and one or more analysis devices 100 can be configured to provide outputs to the processing circuitry as further disclosed herein. In the depicted example, monitoring module 58, reactor module 56, separator 60, analysis module 80, valve module 74, and the analysis device 100 are all communicatively coupled to control module 125, thereby allowing for direct monitoring of various reaction conditions and characteristics as the reaction occurs. However, in other exemplary configuration, as few as one module may be in communication with the processing circuitry. Optionally, it is further contemplated that the control module 125 (alone or in combination with other processing circuitry or a remote computing device as disclosed herein) can be configured to selectively adjust operation of at least one module (e.g., a process module (reactor 56, separator 60) or a regulator module (valve 74)) to optimize the chemical reaction. Exemplary characteristics and conditions that can be optimized using the disclosed feedback loops include, for example and without limitation, one or more of pressure, temperature, an identification of generated products, reagent consumption rate, identification of side products, product yield, selectivity, and purity.

In exemplary aspects, at least a portion of the plurality of modules can cooperate with at least a portion of the plurality of flow components to produce a first configuration that forms a first fluid flow pathway for performing at least one step of a first chemical reaction. After completion of the first chemical reaction, the plurality of modules and the flow components within the substrate layer can be configured for selective rearrangement to a second configuration within a minimal changeover period to produce a second fluid flow pathway for performing at least one step of a second chemical reaction. In these aspects, it is contemplated that the second configuration of modules and flow components can include at least one module that did not define a portion of the first fluid flow pathway. It is further contemplated that the modules and flow components that define the second fluid flow pathway can comprise at least a portion of the modules and flow components that defined the first fluid flow pathway. It is still further contemplated that the number of modules included in the second fluid flow pathway can be less than, equal to, or greater than the number of modules included in the first fluid flow pathway. Optionally, in exemplary aspects, the locations of the plurality of modules and the plurality of flow connectors with respect to the substrate (and manifold layers) can remain unchanged in the first and second fluid flow pathways. In these aspects, it is contemplated that the first fluid flow pathway can be modified by changing flow positions within valves (but not adjusting the mounted position of the valve module with respect to the substrate) to thereby adjust the flow pathway. Optionally, such modifications can allow for bypassing portions of the first fluid pathway (e.g., process modules) and/or directing fluid to other modules (e.g., process modules) that were previously not in fluid communication with the first fluid flow pathway. Although not required, in some optional aspects, it is contemplated that modules can be removed, added, or replaced to selectively adjust the fluid flow pathway. Thus, in some exemplary aspects, the modified second fluid flow pathway can be produced by adjusting fluid flow within a valve module and removing, adding, or replacing at least one module of the system. With the addition or removal of modules as disclosed herein, it is contemplated that the position and/or number and/or type of flow connectors can be adjusted to accommodate the change in the fluid flow pathway.

In further exemplary aspects, it is contemplated that the minimal changeover period can permit sequential performance of multiple chemical reactions in a limited time window that is far smaller than possible with conventional reaction structures. Optionally, the minimal changeover period can range from about 30 minutes to about 4 hours or, more typically, from about 1 hour to about 2 hours, depending upon the complexity of the reaction.

Optionally, the disclosed system 10 can comprise a plurality of regulator modules 64. In exemplary aspects, it is contemplated that the first and second configurations of the plurality of modules and the plurality of flow components can comprise respective first and second arrangements of regulator modules, with the first and second arrangements of regulator modules differing from one another with respect to at least one of module positioning and type of modules. Optionally, in some exemplary aspects, it is contemplated that each arrangement of regulator modules can comprise at least five of the following: a check valve, a tee filter, a flow regulator, a pressure sensing module, a pressure relief valve, a pressure regulator, a tube adaptor, a valve, a pump, a control valve module, a temperature monitoring module, a temperature control module, a heater, or a cooler. Optionally, in these aspects, the second configuration can include at least one module type that is not present in the first configuration. It is further contemplated that the second configuration can include more or fewer regulator modules than were included in the first configuration.

In further exemplary aspects, it is contemplated that the disclosed system can permit performance of multiple or separate reaction steps simultaneously. For example, in one exemplary application, separate products or byproducts from a process module (e.g., a separator module after a separation step) can be delivered to distinct modules (and separate downstream flow paths) for further analysis and/or processing (reaction, separation) as disclosed herein.

Optionally, the disclosed system 10 can comprise a plurality of analysis devices. In exemplary aspects, it is contemplated that a first configuration of the plurality of analysis devices can be in operative communication with the first fluid flow pathway, and the plurality of modules and the flow components within the substrate layer can be configured for selective rearrangement to establish operative communication between a second configuration of the plurality of analysis devices and the second fluid flow pathway. In these aspects, it is contemplated that the first and second configurations of the plurality of analysis devices can include at least two of the following: a UV-Vis spectrometer, a near-infrared (NIR) spectrometer, a Raman spectrometer, a Fourier Transform-Infrared (FT-IR) spectrometer, a nuclear magnetic resonance (NMR) spectrometer, or a mass spectrometer (MS). Optionally, in these aspects, the second configuration of the analysis devices can include at least one analysis device type that is not present in the first configuration. It is further contemplated that the second configuration can include more or fewer analysis devices than were included in the first configuration.

In one example, and as shown in FIG. 6, it is contemplated that the disclosed system can define alternative flow pathways that can be selectively put in fluid communication with process modules (e.g., reactors 56) using various valve modules 74. As shown, it is contemplated that the valve modules 74 can be used to selectively modify the flow pathway and direct flow to a first reactor during a first configuration while directing flow to a second, different reactor during a second configuration. Still further, the valve modules 74 can be positioned to provide for a complete bypass of at least one process module (e.g., reactors 56 as shown in FIG. 6).

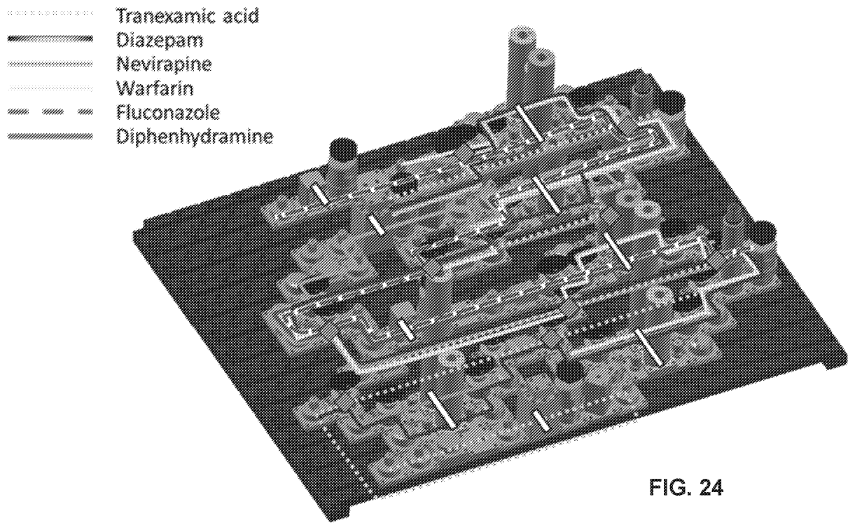

In another example, and as shown in FIG. 24, it is contemplated that a single arrangement of surface-mounted modules as disclosed herein can be used for a series of synthetic routes for different compounds. In this specific example, alternative synthetic routes for tranexamic acid, diazepam, nevirapine, warfarin, fluconazole, and diphenhydramine are shown. As shown, it is contemplated that the depicted arrangement of modules can support numerous potential flow pathways, which can be varied based upon the particular modules that form the flow pathway. In this particular example, it is contemplated that valve modules and manifold flow connectors can be used to selectively vary a fluid flow pathway to permit performance of multiple reactions using a single surface-mount module configuration. It is further contemplated that residence times within particular modules can be selectively adjusted to permit further variation in synthetic routes.

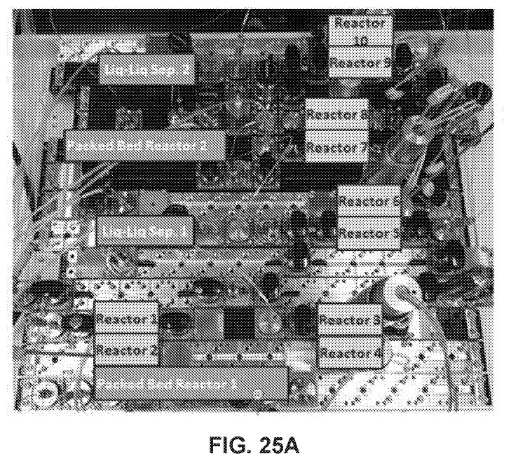

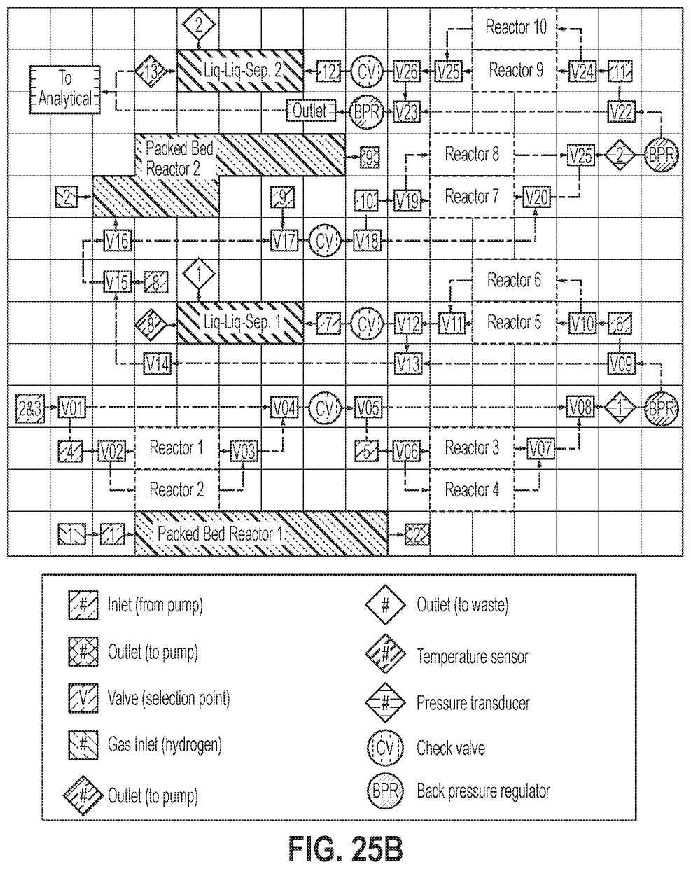

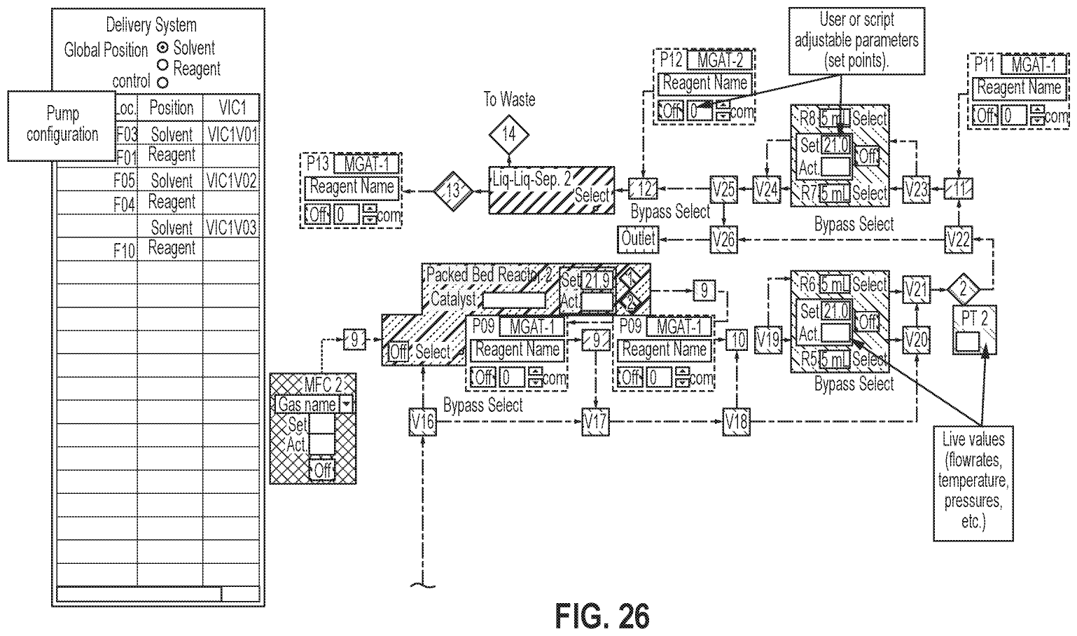

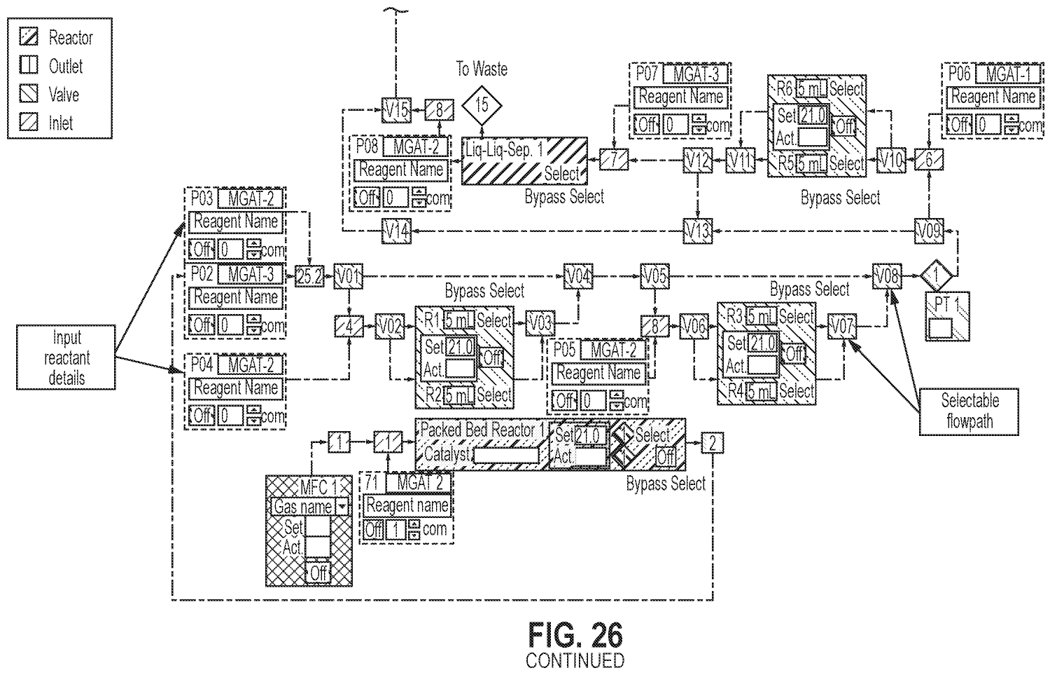

FIGS. 25B-32 show various schematic diagrams depicting various system configurations as disclosed herein. As shown in FIG. 26, it is contemplated that the disclosed system can receive information from various user interfaces at various locations throughout the system. For example, in some exemplary aspects, it is contemplated that a user can use processing circuitry having user interfaces as disclosed herein to manually adjust or use a script to adjust various parameters (set points) within the system, which in turn can optionally be used to adjust operation of the system components as further disclosed herein.

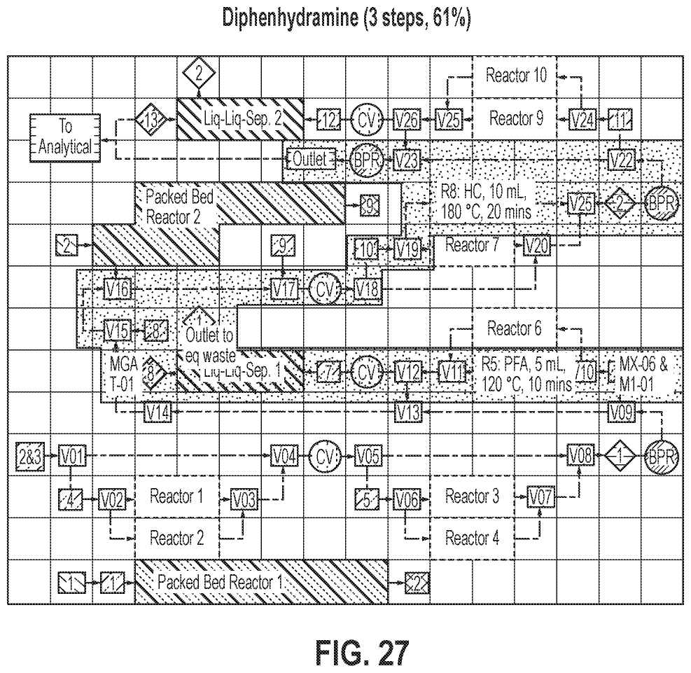

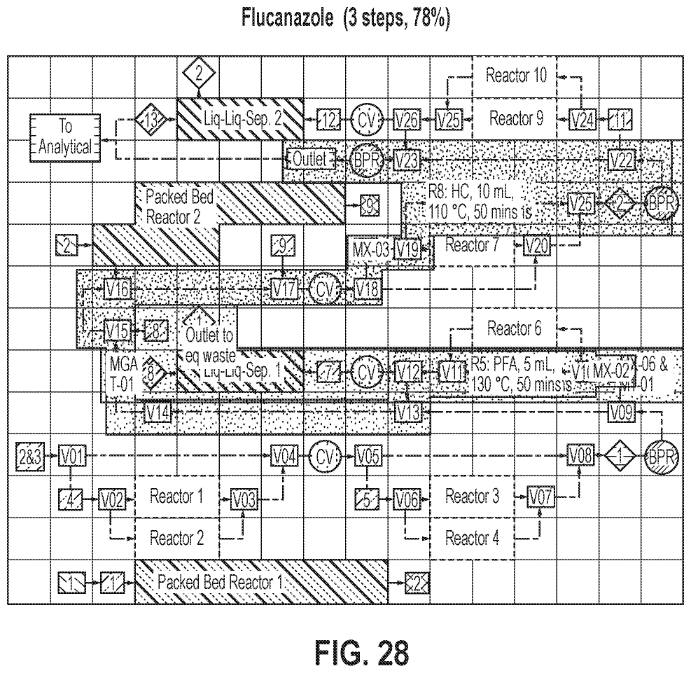

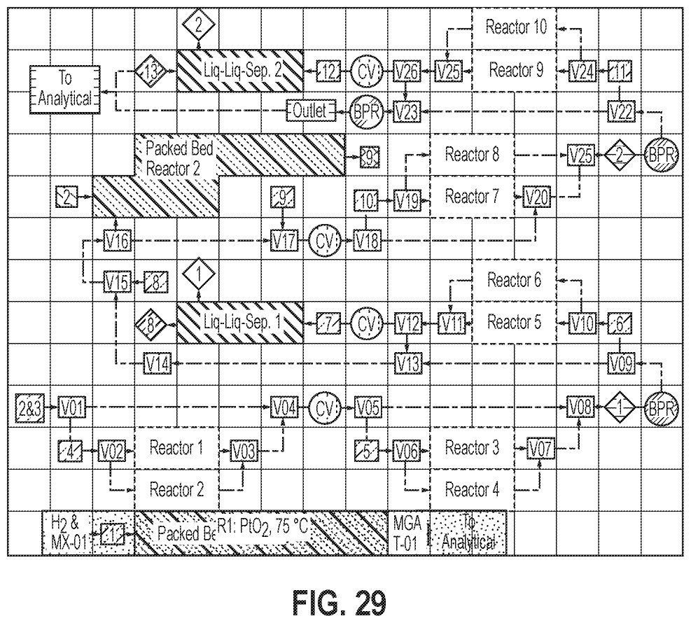

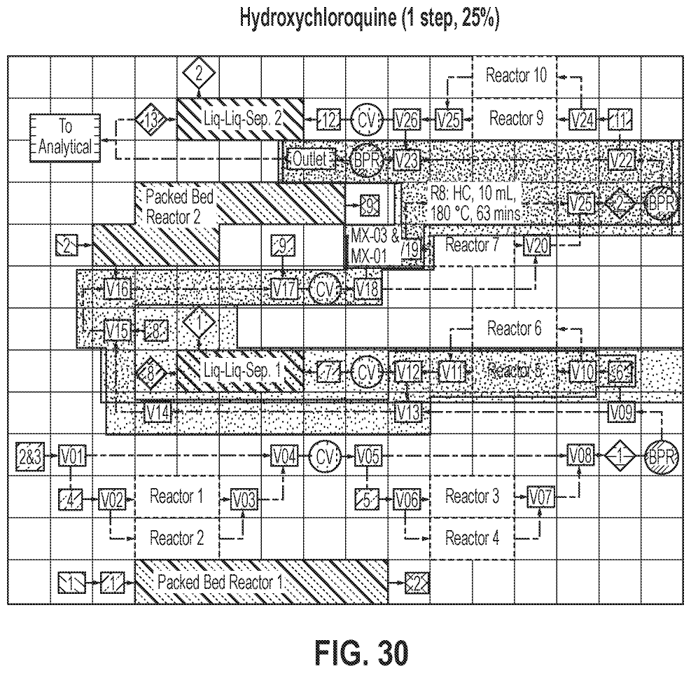

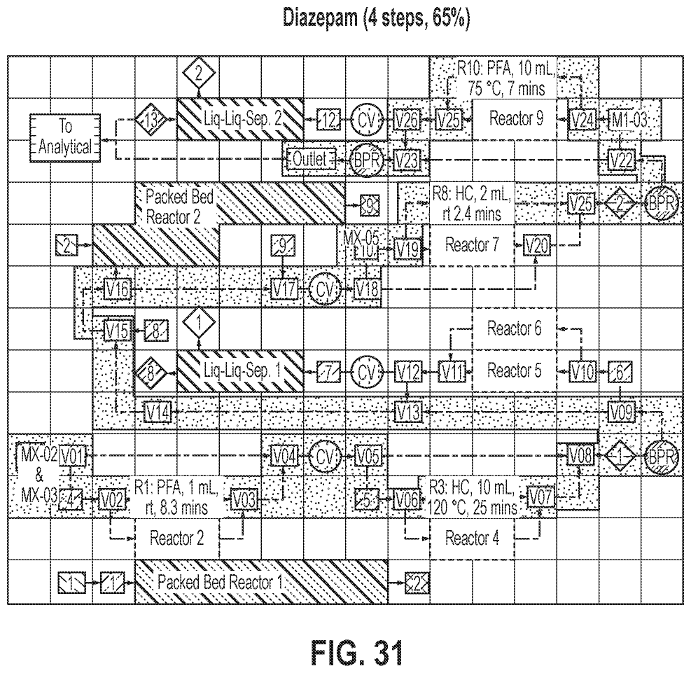

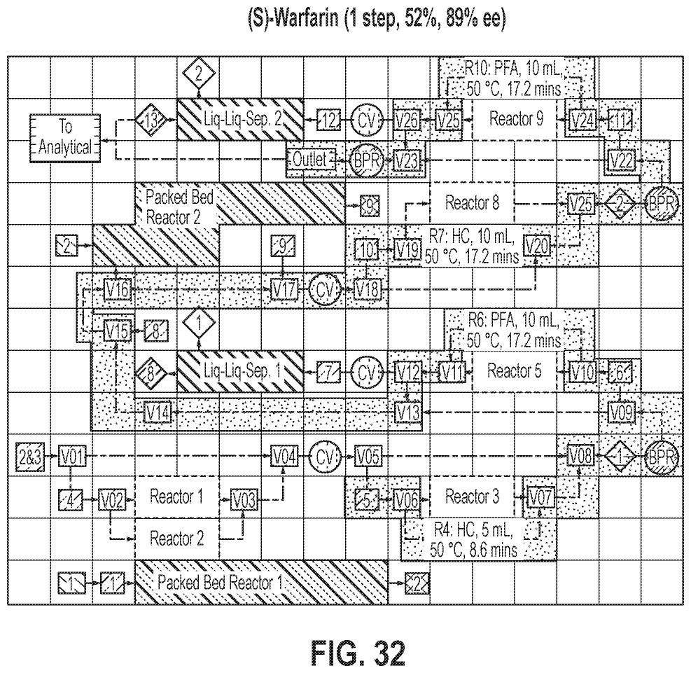

FIGS. 27-32 depict a single surface-mounted module configuration for performing different reactions to produce different reaction products. Each figure highlights the actual flow pathway that was used to perform the indicated reaction. As shown, while FIG. 27 depicts the flow pathway passing through a first liquid-liquid separator, the flow pathway of FIG. 28 bypasses the first liquid-liquid separator. FIGS. 29-30 demonstrate the use of the same module configuration to perform different reactions using only a small number of the modules. FIGS. 31-32 depict more extensive fluid flow pathways, with the flow pathway of FIG. 31 passing through Reactor 1, Reactor 3, and Reactor 8, while the flow pathway of FIG. 32 bypasses Reactors 1, 3, and 8 and passes through Reactors 4, 6, and 7 (which were bypassed by the flow pathway of FIG. 31).

### Methods

An exemplary method of using the disclosed systems can comprise introducing at least one reagent (e.g., liquid reagent) into the fluid flow pathway of the system and then performing a chemical reaction using the at least one reagent (e.g., liquid reagent).

Optionally, in some aspects, the at least one process module comprises a plurality of process modules, and the chemical reaction can be a multi-step chemical synthesis comprising a plurality of sequential steps. In these aspects, it is contemplated that each step of the plurality of sequential steps can correspond to flow of reagents within a respective process module.

In further aspects, the method can comprise modifying the fluid flow pathway to produce a second fluid flow pathway different than the first fluid flow pathway as disclosed herein. As further described herein, the second fluid flow pathway can be different from the first fluid flow pathway in: number of flow modules, number of monitoring modules, location of monitoring modules, number of process modules, type of process modules, sequence of process modules, location of process modules, number of regulator modules, type of regulator modules, location of regulator modules, number of analysis modules, location of analysis modules, direction of flow, and combinations thereof. Further, the method can comprise running a second chemical reaction using a modified fluid flow pathway including the additional process module.

Optionally, the modification of the first fluid flow pathway can comprise adjusting the flow of liquid through at least one valve module among the plurality of modules without the need for adjusting the position of any module relative to the substrate layer (or manifold layer). Optionally, it is contemplated that the fluid (e.g., liquid) flow path of the chemical reaction can be adjusted using valves without the need for adjusting the positions of the surface-mounted components and/or the positions and orientation of flow connectors as disclosed herein. Additionally, or alternatively, in other aspects, the modification of the first fluid flow pathway can comprise mounting an additional process module to the outer surface of the substrate. In these aspects, it is contemplated that the additional process module can be a reactor or a separator as disclosed herein. The method can further comprise establishing fluid communication between the additional process module and the fluid flow pathway.

In further aspects, the method can comprise using the processing circuitry as disclosed herein to receive at least one output from the at least one analysis device. In these aspects, the method can further comprise using the process circuitry to adjust operation of at least one module, such as a process module or a regulator module, to optimize the chemical reaction. Additionally, or alternatively, the method can comprise using the processing circuitry to receive at least one output from a monitoring module as disclosed herein (e.g., a process module or a regulator module equipped with a sensor). The method can further comprise using the processing circuitry to adjust operation of at least one module based upon the received at least one output to optimize the chemical reaction. Optionally, the monitoring and optimization of the chemical reaction can occur at locations within the system corresponding to intermediate steps in the chemical reaction. It is further contemplated that monitoring and optimization of the chemical reaction can take place as the reaction occurs.

As further disclosed herein, it is contemplated that monitoring modules and analysis modules can be selectively positioned at various positions along a reaction flow pathway depending upon the particular reaction steps/locations and conditions/characteristics that a user wishes to monitor.

In further exemplary aspects, it is contemplated that the disclosed system can function as a fully integrated platform for running and modifying chemical reactions. Optionally, each of the modules of the system can be communicatively coupled to the computing device 120, which can be used to monitor and adjust each of the modules within the system based on feedback from analysis tools, including software executed by the processing unit 122. In exemplary aspects, and as further disclosed herein, the system 10 can comprise a user interface for entering instructions for configuring a chemical reaction, and the processing unit can be configured to determine the appropriate modifications to achieve the selected configuration and to then effect automated modification of the plurality of modules as required to achieve the selected configuration.

In use, it is contemplated that the disclosed systems can allow for performing multi-step chemical synthesis reactions in a continuous manner not previously achievable. It is further contemplated that the disclosed systems can permit performance of modular liquid flow reactions that are not achievable using other surface-mount reactor systems. It is still further contemplated that the disclosed systems can provide for intermediate processing steps (at an intermediate step in a reaction) in a manner not previously achievable; previously, such processing could only be performed at the end of a reaction sequence. Additionally, it is contemplated that the disclosed systems can provide for reactions using smaller volumes of reagents, shorter residence times, and/or shorter heating times in comparison to previous chemical reactions.

Optionally, in exemplary aspects, it is contemplated that the flow rates within the disclosed system can range from about 0.05 mL/min. to about 40 mL/min. and more preferably range from about 0.1 mL/min. to about 2 mL/min.

Optionally, in exemplary aspects, it is contemplated that the volume of each reactor module disclosed herein can range from about 0.5 mL to about 50 mL and more preferably range from about 2 mL to about 15 mL.

Optionally, in exemplary aspects, it is contemplated that the volume of each gravity-based liquid-liquid separator module can range from about 0.2 mL to about 10 mL and more preferably range from about 1 mL to about 5 mL.

Optionally, in exemplary aspects, it is contemplated that the volume of the gravity-based gas-liquid separator module can range from about 1 mL to about 20 mL and more preferably range from about 4 mL to about 10 mL.

Optionally, in exemplary aspects, it is contemplated that the flow rates within the disclosed system can range from about 0.05 mL/min. to about 40 mL/min. and more preferably range from about 0.1 mL/min. to about 2 mL/min.

Optionally, in exemplary aspects, it is contemplated that the total volume of the disclosed system can range from about 20 mL to about 500 mL.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

In exemplary aspects, the disclosed system can automate and integrate synthetic design method and chemical synthesis steps to create desired molecules in a continuous and scalable process, from starting materials to finished products. In these aspects, the disclosed system can be an open-source, automated multistep synthesis platform that can be used to perform route optimization and production scale-up.

### Example 1

The disclosed system can provide a standardized configuration of flow chemistry unit operation modules that can perform a wide range of chemistries while minimizing the number of modules needed and eliminating user reconfiguration between routes. The disclosed system can comprise a serial arrangement of parallel modules with selector valves for connection and/or bypass between modules. Initially, a single Process Analytical Technologies (PAT) block of various spectroscopic sensors (UV/VIS, NIR, Raman, MS, and FTIR) can be located downstream of the modules that allow for the serial optimization of process steps. A schematic of the exemplary arrangement of the unit operation as used herein is shown in FIG. 4. A stainless steel hydrogenation reactor 56a is continuously connected with a stainless steel separation reactor 60a that is continuously connected with one or more of various stainless steel flow reactors 56b that are continuously connected with one or more acid resistant flow reactors 56c. The acid resistant flow reactors 56c are continuously connected with one or more acid resistant quench or extraction separation reactors 60b that are further continuously connected with an additional set of one or more of stainless steel flow reactors 56d and 56e. The additional set of one or more of stainless steel flow reactors 56e is further continuously connected with an additional set of both acid resistant and stainless steel extraction and separation reactors 60c. It is understood that a sample at the output of each reactor is collected and proceed by various analytical techniques 100, for example inline optical testing or online mass spectroscopy. The sample exiting the analytical testing equipment 100 and the additional set of flow reactors 56e and the additional set of extraction and separation reactors 60c is collected as a product.

### Example 2

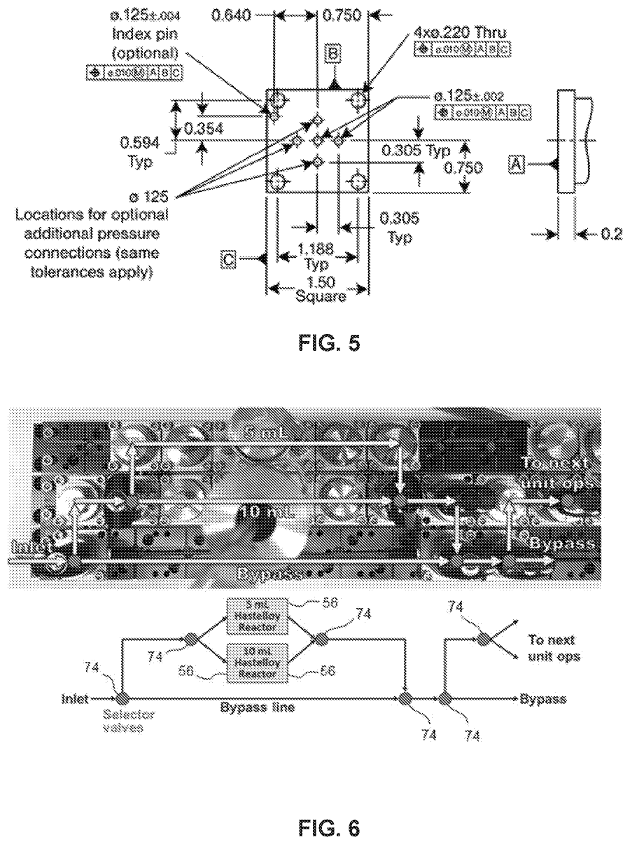

The design of physical components, and specifically dimensional requirements of the components used herein can fall under the SP76 standard, also known as ANSI/ISA-76.00.02-2002 Modular Component Interfaces for Surface-Mount Fluid Distribution Components—Part 1: Elastomeric Seals. This standard, as shown in FIG. 5, can define the location of mounting holes and port connections for surface-mount components and stipulates the use of elastomeric seals, but otherwise allows the architecture to be designed by each manufacturer.

The disclosed system can include three separate layers. A surface-mount layer can include actual components that interact with the flow, such as regulators, valves, sensors, fluid inputs/outputs, etc. These are held in place by #10-32 bolts threaded into the substrate piece. Since the surface mount components are held in place only by these retaining bolts, components can be swapped in place without significant disassembly of the entire system. A substrate layer can include small flow components, which slot into the substrate, act as pipes for transport of the fluid flow, and connect the surface-mount and/or manifold layers together. A commercial off-the-shelf (COTS) design for the substrate layer allows for connection to three separate ports on the surface-mount layer along the same axis as the substrate piece, and the ports can act as either inlets or outlets. These ports are face-sealed with AS568-006 elastomeric O-rings, or AS568-005 PTFE O-rings. Finally, a manifold layer can be similar to the substrate layer but connects in only a single position and runs either perpendicular or parallel to the substrate layer. This allows the flow to be daisy-chained from one substrate to another, sent to parallel blocks, or even to bypass entire sections. Additionally, the bottom of the substrate can be bolted to supporting pieces and feet, which can be mounted to a platform for additional support of the assembled system.

As further disclosed herein, in addition to the surface-mount components provided in the commercially available system, the disclosed system can further comprise at least one process module, such as a reactor or a separator. It is further contemplated that the disclosed system can comprise monitoring modules and/or analysis modules that are not available in commercially available systems.

### Example 3

An exemplary baseline arrangement for the standardized configuration of unit operation modules can include a tubular reactor PFA, a tubular reactor HO, inlets/mixing tees, membrane separators, a packed catalyst bed reactor, bypass manifolds, and switching valves.

To direct the flow between different unit operations or a bypass line, selector valves were used. FIG. 6 shows an overhead view of two surface-mounted reactors in parallel along with a bypass and the possible flow paths. Although not shown, these valves can be automated with integrated servomotors and absolute position encoders for control and feedback that can be operated by the software.

### Example 4

To construct an exemplary flow system various flow components were utilized. The base of the disclosed system (i.e., the substrate and manifold layers) can comprise various substrate and manifold channels. Exemplary substrate and manifold channels can be machined from anodized 2024 aluminum alloy with holes for mounting surface-mount components, additional manifolds, and support blocks. Such substrates can contain mounting holes for surface-mount components #10-32↓0.25″, through-holes for mounting manifold channels, through-holes for mounting support blocks, a locating hole for side components, and a locating hole for center and drop-down components.

It is understood that the disclosed substrate channel system is modular and extensible to additional spaces. Multiple substrate channels can be connected via the use of a spacer foot, or longer channels that can be purchased or fabricated. Optionally, an extended substrate channel that repeats the mounting-hole configuration on a 1.53″ distance between centers can be used. Channels up to 14 spaces long are available off the shelf from commercial vendors vendors.

It is understood that the manifold layer can allow the flow to move below the substrate layer. The two possible directions are perpendicular in standard manifold channels, or parallel in parallel manifold channels.

The standard manifold channels can allow for transport of fluid from one substrate channel to one or more other substrate channels. These are frequently used to “daisy-chain” the flow in a back-and-forth manner to create a more space-efficient setup. An exemplary pattern for the manifold layer repeats with 1.60″ between centers. Manifold channels and manifold fold components mount directly to the bottom of substrate channels. The standard manifold layout comprises a mounting hole for attaching manifold channel to a substrate layer and a locating hole for manifold components. Manifold channels are available from commercial vendors with one to ten positions. Parallel manifold channels can transport the flow beneath the substrate channels but be in parallel with the channel that they are mounted to. The parallel manifold channels can comprise a mounting hole for attaching parallel manifold channel to a substrate layer and a locating hole for parallel manifold components. This allows the flow to “jump” a position, effectively bypassing a surface-mount position. The use case for this occurs less frequently than the standard manifold components and is largely restricted to bypassing two-position surface-mount units (surface-mounted modules that occupy more than one standard mounting position), splitting and re-mixing flows, or to make space for a non-fluidic or non-standard sized unit to be mounted to the substrate channel above. Parallel manifold channels are available from commercial vendors with three to six positions. Surface mount components of the disclosed system can utilize commercially available surface-mount components such as on/off valves, switching valves, check valves, flow-through caps, inlets, and inlet tees.

Additional surface-mount components that can optionally be purchased from commercial vendors include 2-way and 3-way ball valves. Like their non-MPC counterparts, these are ¼-turn valves. The pressure rating of these valves is 2500 psig, and the temperature rating is 20-150° F. (−6-65° C.). Wetted components include the CF3M body, 316 ball stem, PFA packing, 300 series side rings/discs, and either a FKM or FFKM side plug seal. The 2-way valves can be used in the disclosed system for shut-off of inlet/outlet streams or isolation, while the 3-way valves can be used for the bypass system disclosed herein.

The system can further comprise compression tube fitting adapters. Such adapters are available from commercial vendors, for example the following adapters can be utilized in the disclosed system: a) 0⅛″ tube fitting, 1-port; b) ⅛″ tube fitting, 2-port; c) ¼″ tube fitting, 1-port; and d) ¼″ tube fitting, 2-port. Use of these adapters permits the use of tubing connections using metal or polymeric ferrules as manufactured by commercial vendors. These adapters also allow the use of tube-end pressure gauges directly on the disclosed system. The 1-port adapter can primarily be used for the first and last blocks for inlet/outlet, while the 2-port adapter can be used to inject/withdraw from a flow stream. These adapters are rated for 3600 psig and a temperature range of 20-300° F. (−6-148° C.), although the compression fitting ends themselves are rated for excess of 10000 psig. Wetted material is the CF3M body. Female National Pipe Thread (FPT) fitting are also available from commercial vendors.

The disclosed system can further comprise flow-through caps. Optionally, such caps can be commercially purchased from commercial vendors. The 0-port caps can be designed to block off unused positions on the substrate channels. The 2-port caps can be designed to provide flow across the channel or to adapt a center-position connector to a side position. These caps are rated for 3600 psig and a temperature range of 20-300° F. (−6-148° C.). Wetted material is the CF3M body.

The disclosed system can further comprise check valves. The check valves are available from commercial vendors. The valves can be purchased as 2-port and 3-port (1-outlet and 2-outlet, respectively, with center inlet for both) configurations. Cracking pressure is 3 psi, and reseal pressure is 6 psi. These check valves are rated for 3600 psig and a temperature range of 20-300° F. (−6-148° C.). Wetted materials are the CF3M body, 316SS poppet and poppet stop, 302SS spring, and FKM/FFKM O-ring.

The disclosed system can further comprise tee filters. These tee filters are available from commercial vendors for light filtration duty. These filters have replaceable filter elements in a variety of pore sizes from 0.5-90 μm sintered filters and 40-440 μm strainer filters. These filters are rated for 3600 psig and a temperature range of 20-300° F. (−6-148° C.). Wetted materials are the CF3M body, 316SS bonnet, 302SS spring, 316L filter elements, and silver-plated gasket. Final number determined by filter element. Sintered elements are available in 0.5 (05), 2 (2), 7 (7), 15 (15), 60 (60), 90 (90) μm nominal pore size. Strainer elements are available in 40 (40), 140 (140), 230 (230), 440 (440) μm nominal pore size.

The disclosed system can further comprise pressure reducing regulators. Such pressure-reducing regulators are available from commercial vendors. The regulators are available with control ranges up to 1500 psig. These regulators are rated for a maximum of 3600 psig and a maximum operating temperature of 176° F. (80° C.). Wetted components are 316SS body, S17400 poppet, 302SS poppet spring, PCTFE seat, and FKM/FFKM seals.

The disclosed system can further comprise back-pressure regulators available from commercial vendors. The back-pressure regulators are available with control ranges up to 250 psig. These regulators are rated for a maximum of 250 psig and a maximum operating temperature of 176° F. (80° C.). Wetted components are 316SS body, seat retainer and piston, PCTFE seat, and FKM/FFKM seals.

The disclosed system can further comprise relief valves available from commercial vendors. In exemplary configurations, it is contemplated that relief valves for low and high pressure, respectively can be utilized. The low-pressure relief valves can use an adjustable spring for the pressure relief range of 10-225 psi. The high-pressure relief valves can use different springs for different pressure ranges. The low-pressure relief valves can have a pressure rating of 300 psig and a temperature rating of 10-275° F. (−12-135° C.). The high-pressure relief valves can have a pressure rating of 3600 psig and a temperature rating of 25-250° F. (−4-121° C.).

Optionally, the disclosed system can further comprise tube adaptors. Exemplary tube adapters are available from commercial vendors and are used for the connection of commercially available tube fittings. These adapters come in a variety of different configurations, either as a 1-port version, which is primarily used for the first and last blocks for inlet/outlet, or as a 2-port version, which can be used to inject/withdraw from a flow stream. These adapters are rated for 3600 psig and a temperature range of 20-300° F. (−6-148° C.), although the compression fitting ends themselves are rated for excess of 10,000 psig. Wetted material is the CF3M body.

The disclosed system can further comprise pneumatically actuated low-pressure valves. Exemplary low-pressure valves are also available from commercial vendors. These valves are available in 2-port and 3-port configurations for on/off control. The valves have a pressure rating of 250 psig and a temperature rating of 0-150° F. (−17-65° C.). Wetted materials include 316L body, UNS R30003 diaphragm (cobalt super alloy), and a PCFTE seat.

The disclosed system can also include a stream selector valve (SSV) system for switching between multiple inlets and a common outlet. Exemplary SSV systems can be purchased from commercial vendors. This SSV can be a double block-and-bleed module that can select from up to 10 different inlet streams. This valve is primarily designed for switching between various sampling streams for a process gas analyzer. The SSV has a pressure rating of 250 psig and a temperature rating of 20-300° F. (−6-148° C.). Wetted materials include CF3M body, 316SS flange and insert, and FKM/FFKM seals.

The disclosed system can further comprise an integrated valve control module (VCM). Exemplary VCMs are also available from commercial vendors for control and monitoring of up to six pneumatic valves. These are DeviceNet-compatible and can function with any valve equipped with a Turck Bi 1-EG05-APEX position sensor.

The disclosed system can further comprise integrated temperature/pressure transducers. Exemplary temperature/pressure transducers are available from commercial vendors. These can be microelectromechanical system (MEMS)-based sensors that can measure pressures up to 500 psig and temperatures from 23-158° F. (−5-70° C.). They are UL certified for use in hazardous locations and use a single M12 connector for power and communication. Wetted materials are 316SS diaphragm and a FFKM O-ring.

It is understood that the fluidic flow path through the system can be determined by the installation of flow components within the substrate and manifold layers. These flow components can act as pipes for transporting the fluid and are assembled by orbital welding of two connector halves together. Each type of connector half connects to a different location on either the surface-mount or manifold layers or slots into the channels using locator pins. Through the use of varying flow components and surface-mount components, the flow path can be designed and built. Additionally, the design of the components is such that they slot into place with ease and cannot be installed incorrectly.

In exemplary aspects, the disclosed system can comprise a surface-mount layer that has three hole positions: two side positions and one center position. Flow components can be designed to either reach the nearest side position or center position. Short (SH) connector pieces can be slotted into either side position and connect to the surface-mount layer. Long (LG) connector pieces can be slotted into central position and connect to the surface-mount layer. Down elbow/manifold (DE) connector pieces can be slotted into the central position and connect to the manifold layer. Down tee/center-manifold (DT) connector pieces can be slotted into the central position and connect to both the surface-mount and manifold layers. In certain aspects, other types of connectors can be used. In some exemplary aspects, a ¼″ compressing fitting (S4) can be utilized. In such aspects, the compressing fitting can be welded to one of the above connectors to provide a convenient side input to a particular surface-mount or manifold location. It is understood that the larger cross section in the middle of the connectors can have a relatively large volume, which results in longer residence times. Additionally, the expansion and contraction zones generate eddies and quiescent zones, which can lead to peak broadening. As one of ordinary skill in the art would readily understand, these factors can negatively impact plug flow operation and the time required to achieve a steady-state condition.

Within the manifold layer (when provided), manifold elbow (ME) connector pieces can be slotted below the central position in the manifold channel and connect the substrate and manifold layers. Manifold tee (MT) connector pieces can also be slotted below the central position in the manifold channel and connect the substrate and manifold layers. Additionally, the manifold tee connector pieces can also tee further into the manifold layer, allowing either mixing or splitting of a flow. Additionally, the manifold layer can include jumper tube connectors which include a SH connector and a LG connector with an extension tube in between. These jumper tube connectors can allow the skipping of surface mount positions.

To mitigate the peak broadening issue observed in the COTS connectors from commercial vendors, the disclosed system can make use of custom variations of the connectors, which maintain the same 1/16″ cross section throughout the connector and lead to reduced peak broadening. The ID of these connectors was well-matched to the tubing used in the disclosed system, providing more consistent plug flow than the COTS connectors from commercial vendors. The connectors having a consistent inner diameter as disclosed herein were manufactured from Hastelloy C-276 and orbital welded.

Several types of tubing can be used for the plumbing of the disclosed system. The primary tubing size used in the system was ⅛″ OD tubing. To create the reaction volumes for the heated/cooled reactors, ⅛″ PFA and Hastelloy C tubing were wound on a mandrel or on a plate. 1/16″ tubing was used sparingly due to the high-pressure drop, and it was used primarily for transfer lines to minimize the swept volume in the system. Allowable pressures and temperatures for various grades of tubing used in the disclosed system are summarized below:

Polymeric Tubing:

⅛″ OD× 1/16″ ID PFA tubing was graded up to 370 psi @ 72° F., −320-450° F.; ⅛″ OD×1.59 mm ID PFA tubing was graded up to 1050 psi; 1/16″ OD×1.00 mm ID PFA tubing was graded up to 800 psi; ⅛″ OD× 1/16″ ID MFA tubing was graded up to 440 psi @ 72° F., −100-485° F.; and ⅛″ OD×0.062″ ID PEEK tubing was graded up to 1000 psi @ 72° F., −320-480° F.

### Metal Tubing:

⅛″ OD×0.028″ wall 316L SS seamless tubing was graded up to 8500 psi; ⅛″ OD×0.035″ wall 316L SS seamless tubing was graded up to 10900 psi; and ⅛″ OD×0.070″ ID Hastelloy C tubing was graded up to 8500 psi. Table 1 shows volume per unit length of various types of flow tubing.

TABLE 1
Volume per unit length of various types of flow tubing.

| Type of tubing | OD (inch) | ID (inch) | uL/cm |
|---|---|---|---|
| ⅛″ SS tubing, standard wall | 0.125 | 0.069 | 24.1 |
| ⅛″ SS tubing, heavy wall | 0.125 | 0.055 | 15.3 |
| ⅛″ HC tubing | 0.125 | 0.070 | 24.8 |
| ⅛″ PFA tubing, 1.6 mm bore | 0.125 | 0.063 | 19.8 |
| 1/16″ PFA tubing, 0.80 mm bore | 0.063 | 0.031 | 4.9 |

Several types of fittings were also used to make the tubing connections. Fittings can be chosen based on a type and size of the tubing. In exemplary aspects, where polymeric tubing was used, either ¼″-28 flat-bottom flangeless fittings, having PEEK nuts and EFTE ferrules, or unions and tees, having EFTE, PFA, and PTFE materials were utilized. In exemplary aspects where metal tubing was used, commercially available compression fittings, with either stainless steel, PTFE, or Hastelloy C-276 ferrules as appropriate were utilized.

As can be appreciated, ANSI/ISA 76.00.02 standard specifies the use of elastomeric seals for sealing surface-mount components. Commercial vendors specify AS568-006 VITON (FKM) 75A durometer O-rings as the basic option for sealing all components (nominal ¼″ OD×⅛″ ID, 1/16″ CS). However, due to requirements for higher operating temperatures and chemical resistance, the disclosed system can utilize Kalrez 7075 (FFKM) seals. Use of Kalrez seals can extend the permissible operating temperature from 400° F. (204° C.) to 625° F. (329° C.) and make the system compatible with a wide range of organic solvents. Additionally, the use of solid PTFE AS568-005 O-rings ( 7/32″ OD× 3/32″ ID, 1/16″ CS) was tested. A smaller sized O-ring was required due to the reduced compressibility of PTFE. These O-rings can also offer an excellent chemical resistance profile, albeit with a reduced maximum operating temperature of 500° F. (260° C.). Table 8 compares the chemical resistance of the various sealing options tested in this invention. Per the specifications of commercial vendors, all #10-32 socket hex head cap screws must be tightened to 10 in-lb (1.13 N-m). The results are grades as: A=Excellent Compatibility; B=Good Compatibility; C=Fair Compatibility; D=Compatibility. Data was collected at room temperature.

To mount the substrate channels in a more permanent scheme, several types of mounting blocks and supports that are available from commercial vendors were utilized. These mounting blocks were affixed to a threaded mount using ¼″ bolts. For this exemplary implementation of the disclosed system, ¼″-20 stainless steel hex socket caps screws were utilized. These blocks bolt onto the end of a substrate channel and provide two holes for ¼″ bolts on a 1″ spacing, and thus allowing the substrate channel to be rigidly mounted to a base plate. As it can be understood this feature is particularly helpful for stabilizing the taller unit operation modules, such as the heated reactors. Also since these blocks are tall enough, they can provide some clearance for the underlying manifold layer.

In exemplary aspects, where the substrate channels have five positions or longer, the support blocks can be used as recommended by commercial vendors. These support blocks can also bolt to a base plate, and thus, increase the rigidity of the substrate channel.

Optionally, for examples where a longer substrate channel is needed, a spacer foot can be used to bolt two substrate channels together. These spacer feet bolt onto the ends of substrate channels like the mounting feet, while maintaining the correct spacing of surface-mounted components. However, and as can be appreciated due to the gap in between, a jumper connector or similar connector can be required to connect one substrate channel to the next.

It was found that when connecting the substrate channels to the mounting feet, an additional lockdown bar can be required when using #10-32×0.50″ screws. Use of the lockdown bar can provide an additional thickness to prevent the screws from bottoming out in the tapped holes. The lockdown bar can also help stabilize the connector when using S4 connector pieces. To further stabilize or permanently install substrate channels, a base panel can be made for attachment of the mounting feet.

For example, the base plate can accommodate 12 substrate channels with up to 14 positions (each in parallel) but still fit inside a standard-depth hood. This pegboard-style plate can allow the combination of virtually any configuration of shorter channels, allowing for testing of entire assemblies individually before installing them into the base plate.

For COTS flow components available from commercial vendors, the following wetted materials were used: 316L SS (ASTM A276 or A479) and fluorocarbon FKM or optional Kalrez. Non-wetted materials used in the system were: aluminum (alloy 2024-T351, hard-coat anodized) and 300 series stainless steel. Custom flow components as disclosed herein were made from Hastelloy C-276.

### Example 5

An exemplary delivery subsystem for the disclosed system used rotary piston pumps available from commercial vendors, which were modified with stiffer piston springs, Viton or Kalrez O-rings on the positions, a stainless steel stator support, and standoff between pump and motor for thermal isolation. The pumps utilized commercially available modules for computer control of the pump stepper motors.

To further extend the chemical resistance and working pressure of the pumps used in the system, a modified stator was designed.

It was found that the Valcon composite that forms the commercial available stator is primarily composed of PPS, with additional PTFE, carbon fiber, and graphite to increase lubricity and stiffness. In regular usage, particularly with strong mineral acids, these stators can exhibit premature wear, causing leakage during operation. In an effort to mitigate this issue, a modification for these pumps was fabricated. An exemplary stainless steel cap can include a press-fit PTFE wetted section. This modification was made to reinforce the PTFE section to allow it to be used as a stator. Glass-reinforced PTFE can be also used instead of virgin PTFE. Without being bound by the theory, it is contemplated that this modification can allow pumping of highly concentrated acids and bases that are not normally compatible with commercially available pumps by leveraging the high chemical resistance of the PTFE material combined with the stiffness of the surrounding stainless steel.

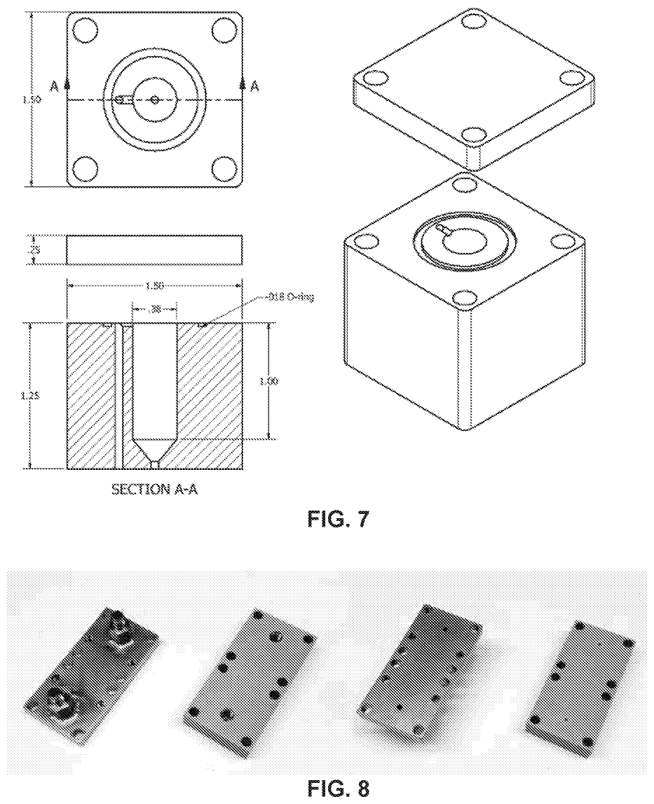

For instances in which flows may not be perfectly balanced, i.e., during startup or flow-rate changes and adjustments, it may be necessary to buffer the flow. To accomplish this, a small surface-mounted holdup tank can be used to provide a small reservoir of liquid. This can be critical due to the need to prevent gas from filling either the pump or the reactors, as this can negatively affect flow consistency and can cause the pumps to lose their prime. A preliminary design for a 2-mL holdup tank is shown in FIG. 7.

### Example 6

The disclosed system can further comprise flow reactors. The flow reactors can optionally comprise tubing around a centrally heated mandrel that is mounted on a stainless steel or PEEK base plate that is surface-mounted onto a substrate layer as disclosed herein. The baseline configuration can utilize ⅛″ Hastelloy or PFA tubing around an aluminum mandrel. To achieve different volumes, different lengths of tubing can be used as well as mandrels with different heights to support the tubing. A thermocouple or RTD can be used to monitor temperature in the mandrel and connect with the control hardware to control the temperature via a cartridge heater inserted into the central bore. Reactors can be insulated using rectangular clamshell insulators that comprise an outer aluminum enclosure and inner rigid ceramic or calcium silicate insulation. The reactors can be further decoupled from the aluminum substrate channel through the addition of a phenolic spacer beneath the base of the mandrel. In addition, the fluid connections for the reactors can vary depending on the type of material used for the reaction tubing. The photographs of exemplary reactor bases are shown in FIG. 8.

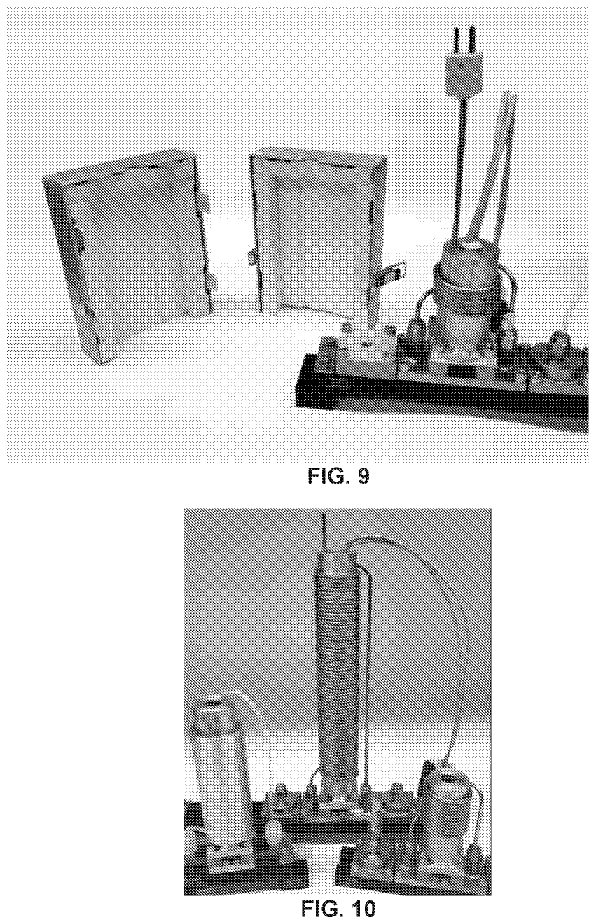

The largest component of the heated flow reactors used in the disclosed system is the clamshell insulator as shown in FIG. 9. The insulators can be slightly larger than the reactor bases, which precludes the use of several reactors in a high-density assembly. However, the disclosed system can include parallel reactors for selection of residence times. Since only one of these reactors should need to be at temperature during a particular run, the disclosed can use insulators that enclose both reactors simultaneously. It is believed that further improvements can be accomplished by reducing the amount of insulation to further shrink the profile of the reactors, and thus, allowing greater flexibility of placement.

The reactors used in the disclosed system can be manufactured with a wide variety of residence times. Exemplary reactors with varying residence times are shown in FIG. 10. Tested volumes ranged from 1 to 10 mL. The metal coil reactors (316SS, Hastelloy) were formed on a separate mandrel and are self-supporting on the heating mandrel. To support the PFA coils, an aluminum tube of 1.625″ OD×1.5″ ID can be used.

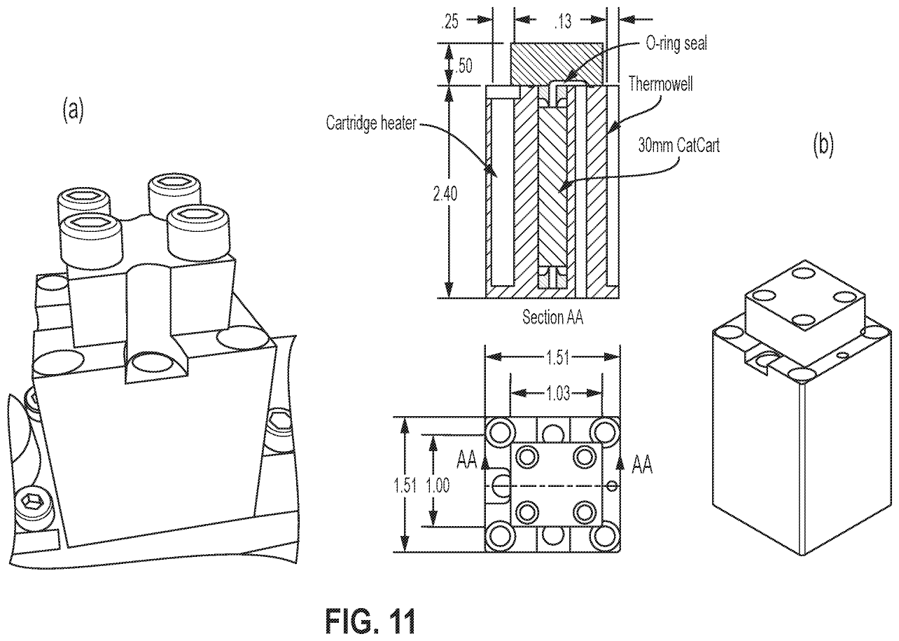

For catalyzed reactions, a method for introducing liquid flow to a solid catalyst was developed. A packed-bed reactor (FIG. 11) was designed to make use of commercially available catalyst cartridges. The reactor can be surface-mounted onto the disclosed substrate layer can be oriented for either downward or upward flow through the column. The cartridge can be replaced by unscrewing the top cap. Three wells can allow for the addition of cartridge heaters, and an additional thermowell can allow the use of ⅛″ diameter temperature probes. The initial design was done for 30×4-mm cartridges, but the height of the design can be increased to handle 70×4-mm cartridges. The prototype reactor was made out of stainless steel, but Hastelloy can be used if increased chemical compatibility is needed.

A wide variety of commercially available catalyst cartridges exist to facilitate a broad spectrum of reactions. They are commercially available with common precious metal catalysts (Pt, Pd, Au, Ir, Rh, Ru, Os) as well as some non-precious metals (Cu, Ni, Co, W, Zn, Fe, S) and specialty cartridges (enzymatic, inert, ion exchange, organic, scavenging). The design of the cartridges allows for rapid replacement and consistent catalyst load from reaction to reaction. Also available are tools for packing custom catalyst cartridges.

The commercially available catalyst cartridge packages can comprise a plastic-sealed cartridge with frits on both ends. This can allow liquid movement through the catalyst bed without entraining solid material. An optional high-temperature version of these catalyst cartridges is available, which utilizes graphite column ends for high-temperature sealing.

### Example 7

It is understood that certain reactions require cooling of the system, i.e., to prevent exothermic runaway, thermal degradation, or side reactions. As one of ordinary skill in the art would readily appreciate, generally, lower temperatures generated by wet ice or dry ice baths. However, this approach limits the operational temperature to the temperature of the cooling media. Mixed-salt baths can also be used to tune the temperature, but otherwise these passive methods have no control mechanism. To actively cool the system, cooling reactors utilizing thermoelectric coolers can be used. Optionally, the cooling flow reactor can be configured for surface-mounting to a substrate as disclosed herein.

Additionally, to cool the reaction directly at the point of mixing, where the exotherm is expected to be highest, a cooled pre-mixer can be used. Optionally, the cooled pre-mixer can be configured for surface-mounting to a substrate as disclosed herein.

### Example 8

In exemplary aspects, the disclosed system can comprise a membrane-based liquid-liquid separator that is configured for surface-mounting to a substrate layer as disclosed herein.

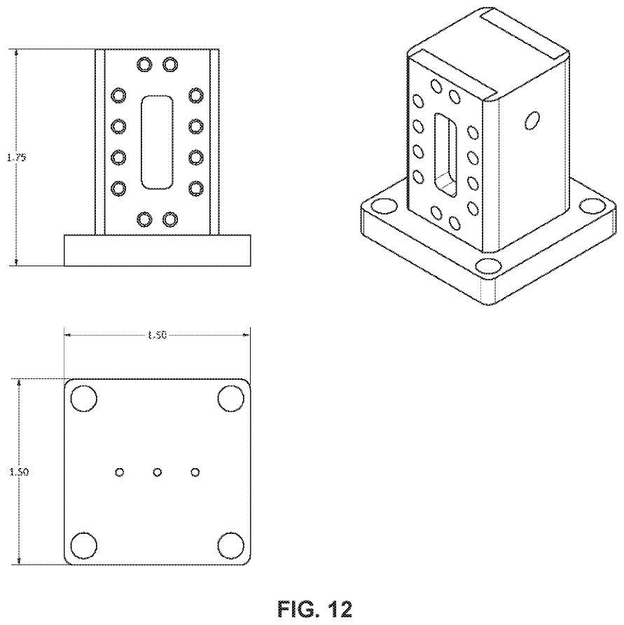

In some other exemplary aspects, separators other than membrane separators can be utilized. For example, it is contemplated that the disclosed system can comprise a gravity-based liquid-liquid separator as shown in FIG. 12. It is contemplated that the gravity-based liquid-liquid separator can be configured for surface-mounting to a substrate layer as disclosed herein. The exemplary separator design is based on the principle of phase separation by density, similar to a separation funnel. However, unlike a separation funnel, this unit is designed to be operated continuously under pressure.

In additional aspects, the exemplary gravity-based separator can be machined out of virgin PTFE and borosilicate glass. The external retaining components can be made of 6061-T6 aluminum and 18-8 stainless steel. The wetted materials were chosen for their high chemical resistance as well as their surface tension properties. The PTFE surfaces can be wetted by the organic solvent, while the glass can be wetted by the water, which facilitates transport of droplets to the correct phase. Additionally, the separator can have an internal hourglass shape designed to generate a quiescent zone for each phase, giving more time in a laminar settling condition to enhance separation. This additional volume also allows the separator to be run at a larger range of flow rates without entrainment of biphasic mixtures.

The flow can be introduced from the side of the separator to the mid-section of the wetted area. This exemplary design can require active pumping from one of the flow streams in order to maintain the steady state. The first-guess approximation can be taken from the inlet flow volume of the phase being removed, i.e., if performing a water-wash injection after a reactor, the outlet pump rate can be set at same level as the water inlet. In some aspects, flow rates can be changed manually as needed in order to maintain the organic-aqueous interface within the separator. In other aspects, this could be automated with optical or capacitive feedback.

It is contemplated that the disclosed reactor/separator designs can assist in automation of the processes disclosed herein by providing means for feedback between the reactors/separators and central processing components. In certain aspects, to increase the chemical resistance of the wetted metal surfaces, various coatings and/or other materials can be utilized.

### Example 9

As one of ordinary skill in the art can appreciate, presence of gases in-line can cause a challenge to consistent flow due to the rapid expansion after a pressure-reducing regulator, which causes the flow to spurt. Without being bound by a theory it is believed that this problem results from the high compressibility of gases compared to liquids. In some aspects, to handle this issue, a gas separation module can be required. For example and without limitation, these modules can be required for separation of permanent gases flashed out after a pressure drop, or to remove gases as they are formed from a reaction.

### Example 10

The disclosed system can use a multifaceted approach to address the online reaction feedback and control provided by process analytical technology (PAT) instrumentation. The instruments were strategically chosen to provide broad characterization of the reaction products and intermediate steps with low instrumentation footprints and high-fidelity data. COTS instrumentation is used due to availability, performance, and capability for support and scale-up. The instruments used in the disclosed system fall under the following three categories: (a) in-line instruments (UV/Vis, NIR, and Raman); (b) in-line/on-line instruments that can be optionally operated offline (FTIR and MS), and (c) off-line instruments used to support development work.

In-Line Instruments

In exemplary aspects, a commercially available UV/Vis spectrometer was utilized as an in-line instrument to provide analytical capabilities for optical rotation dispersion analysis and for verification of aromatic and conjugated species. A fiber optic interface, with a typical optical range of 200-1100 nm, provides the capability for remote standoff of the instrument from the fluid flow pathways defined by the flow connectors and surface-mount components as disclosed herein. The fiber probes have been adapted to a custom, low-volume, stainless steel/Kalrez sampling cell. Data can be typically acquired at 1 sample in 2-5 s, although faster acquisition is possible when desired. In exemplary aspects, the data files are acquired and stored using commercially available software.

In other exemplary aspects, the a commercially available NIR spectrometer can be used for solvent detection and identification, especially in the plug-flow approach for sample screening. Similar to the UV/Ms above, the optical fiber probes for the NIR spectrometer can be coupled to a custom, low-volume, stainless steel/Kalrez sampling cell. In exemplary aspects, data is acquired using commercially available software.

In still further exemplary aspects, a commercially available Raman spectrometer can be coupled to a laser and used in conjunction with FTIR for vibrational and fingerprint analysis. Samples can be collected, and conventional processing can be applied to the output data to reduce background fluorescence initiated by the laser light. This design allows for extraction of the characteristic vibrational frequencies and identification of aromatics even when the Raman signal was far smaller than the fluorescence. A custom stainless steel/Kalrez cell can be used for the laser source and detector collection.

In yet other exemplary aspects, a DART (Direct Analysis in Real Time)-MS was used for measurements in the disclosed system. The DART ionization source can provide for rapid, non-contacting sampling. The MS can provide broad selectivity for chemical analysis based on molecular masses and fragmentation and has the potential for MS-MS capability that could aid unknown identification. In other exemplary aspects, it is contemplated that other instrumentation, including Liquid Chromatography (LC)-MS instrumentation, can be used in a similar manner to DART.

One of ordinary skill in the art would readily appreciate that as the benchmark analytical technique in synthetic organic chemistry, NMR spectra are routinely measured to validate flow synthesis experiments. In general, flow synthesis fractions are concentrated and processed as routine organic chemistry NMR samples (8-32 scans around 1-2 seconds each) in deuterated chloroform or DMSO as appropriate. Sample volumes are between 0.3 and 0.6 mL and typically contain 10 mg of sample. Data can be processed in commercially available software and archived for later analysis.

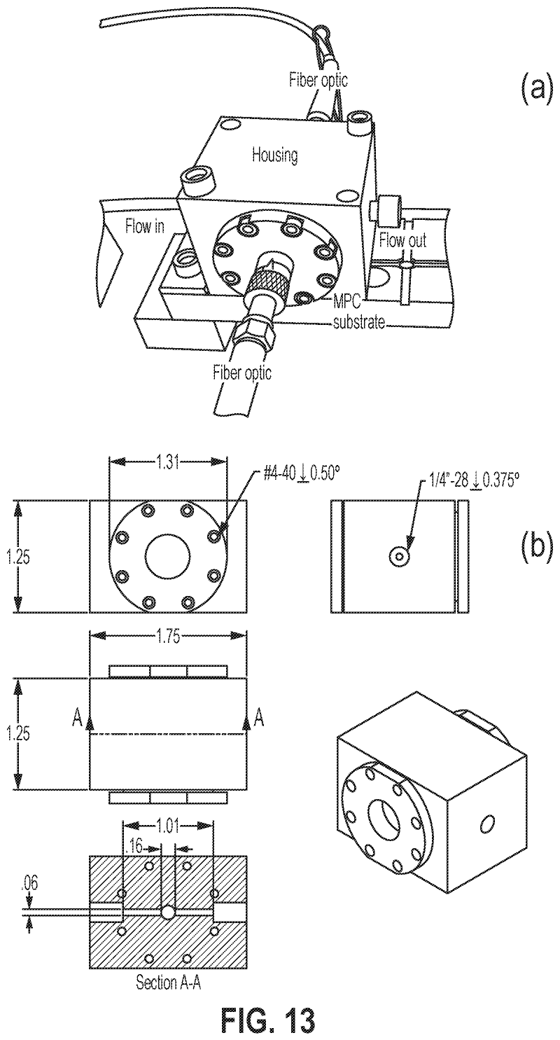

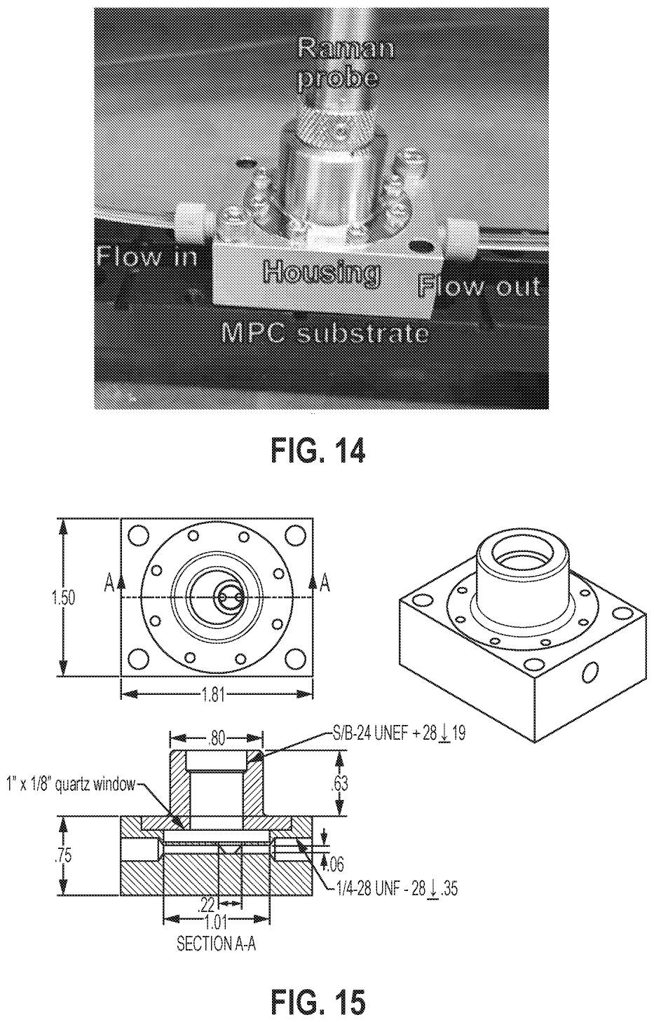

The UV/Ms and NIR flow cells can have a similar design for use with standard SMA 905 fiber optic assemblies. By using custom-machined quartz windows with Kalrez O-rings for sealing, a low swept volume of 85 μL with zero dead volume and a path length of 2.38 mm can be achieved. Additionally, the windows can be easily removed for cleaning if they become fouled. A schematic for the UV/Ms and NIR cells is shown in FIG. 13. The Raman flow cell interfaces with an immersion-ready RPR probe. This probe has a stainless steel sampling head and Hastelloy sleeve. In some exemplary aspects, the probe was interfaced with the flow through an ⅛″ quartz window parallel to the flow as shown in FIG. 14 and FIG. 15.

In-Line Instruments Optionally Operated Off-Line

In exemplary aspects, the FTIR was used to provide in-depth functional groups and vibrational fingerprinting. An FTIR instrument is amenable to in-line monitoring. In some exemplary aspects, in-line FTIR was optionally used off-line. In such aspects, samples were collected in glass vials and then transferred to the FTIR spectrometer. There is often some solvent loss with this interface. Analysis was carried out with commercially available software. Comparison measurements are taken between standard samples of pure compound and the outputs of the disclosed platform.

In some exemplary aspects, the instrumentation was used off-line in some combination of OpenSpot cards (IonSense) or liquid sampling as further described herein.

### Example 11

In this example, diphenhydramine was synthesized using the disclosed system The single-step modules—such as flow reactors and separators—that can be connected to build multistep processes were designed as described above. Reaction output of diphenhydramine was measured by a bank of process analytical technology (PAT); off-line analyzers such as DART-MS, FTIR, and NMR (however, it is contemplated that such analyzers can also be configured for on-line use as further disclosed herein).

Solvents were from Macron Fine Chemicals and reagents were from Sigma-Aldrich. Tubular reactors were manufactured in-house from 1/16″ ID PFA tubing, 0.069″ ID stainless steel tubing, or 0.070″ ID Hastelloy. Commercially available pumps were used to pump reagents and solutions. Pressure was controlled with varying 250 psi back pressure regulators. Commercially available check-valves were used. Reagent streams were combined using PFA T-mixers with 1.00 mm ID. The liquid-liquid extractor was also constructed in-house with a commercially available PTFE body, a commercially available 0.5 μm PTFE membrane, a commercially available 0.002″ PFA diaphragm, and pressed between two stainless steel plates. Yield and ratios were determined by NMR.

3-Step synthesis of diphenhydramine was done according to the following process. The preparation of diphenhydramine was developed from benzophenone. Commercially available pumps were primed with benzophenone (1.51 M in toluene) and DIBAL-H (1.53 M in toluene). A 5 mL PFA reactor manufactured in-house was maintained at room temperature (22° C.). Benzophenone and DIBAL-H were flowed at 0.250 mL/min (t_{R}=10 minutes). Then the reduction mixture was connected to a commercially available check-valve to prevent reverse flow from the subsequent introduction of HCl (10.0 M, aq) from a 3rd pump flowed at 0.500 mL/min. The chlorination mixture was flowed into a 10 mL PFA reactor heated to 120° C. (t_{R}=10 minutes). A short segment of PFA tubing led to a 6 bar acid-resistant back pressure regulator. The reaction mixture was separated at ambient pressure into a holding reservoir. Pumps flowed the resulting chlorodiphenylmethane (0.755 M in toluene) and 2-dimethylaminoethanol (9.93 M, neat) at 0.167 mL/min into a 5 mL Hastelloy reactor heated at 180° C. (t_{R}=15 minutes). The reaction mixture was then connected to a commercially available, varying 250-psi back pressure regulator. Six fractions were collected every 15 minutes. Post-synthesis, each fraction undergone an aqueous work-up to remove excess 2-dimethylaminoethanol. Diphenhydramine was washed several times with water and brine. The organic layer was dried with sodium sulfate and concentrated. ¹H NMR matched the values reported in literature. ¹H NMR (400 MHz, CDCl₃) δ 7.36-7.29 (m, 8H), 7.25-7.23 (m, 2H), 5.37 (s, 1H), 3.57 (t, J=6.4 Hz, 2H), 2.60 (t, J=6.4 Hz, 2H), 2.27 (s, 6H).

Results of the formation of diphenhydramine using the disclosed system through a 2-step process are shown in Table 2 below.

TABLE 2
2-Step Process

| Fraction | Equiv. HCl | Composition (¹H NMR) | Note |
|---|---|---|---|
| 1 | 24 | 85% DPH (diphenylhydramine) | Equil. |
| | | 15% DPM (diphenylmethanol) | |
| 2 | 24 | 85% DPH, 15% DPM | Equil. |
| 3 | 24 | 93% DPH; 7% DPM | Optimal |
| 4 | 24 | 85% DPH; 15% DPM | Equil. |
| 5 | 3 | 34% DPH; 58% DPM, trace —CL; | Out of spec |
| | | trace ether | |
| 6 | 3 | 44% DPH; 46% DPM; 6% —Cl | Out of spec |

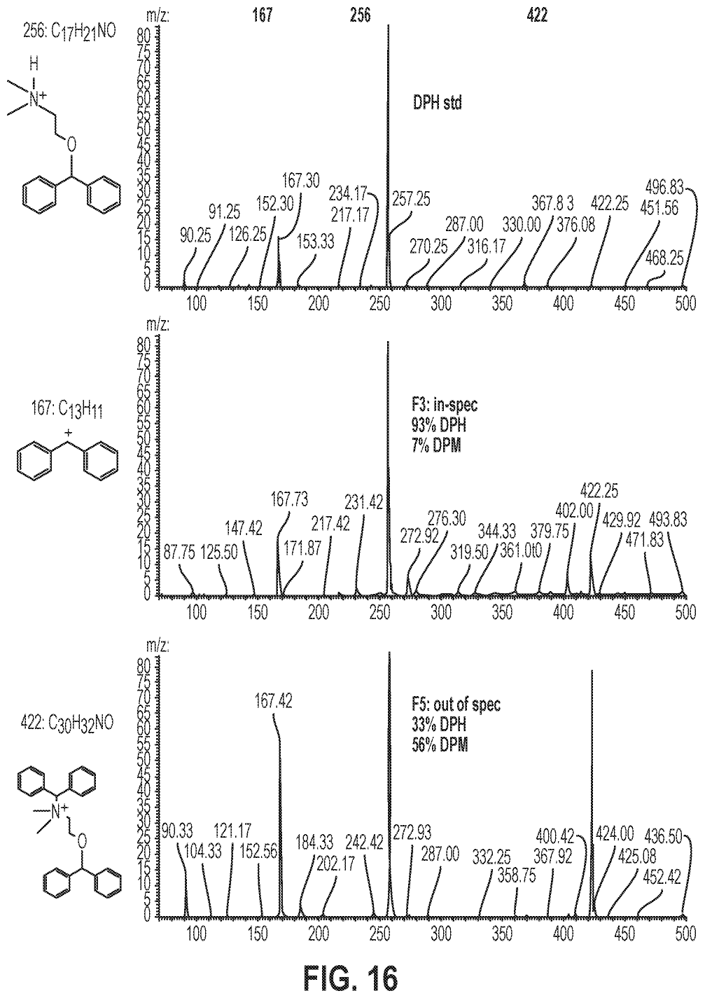

The products of the 2-Step Process were measured using off-line DART-MS analysis and results are shown in FIG. 16. The top panel shows the diphenhydramine standard as run through the system. The middle panel shows the optimal reaction conditions (in-spec, Frac-3, middle panel) with high yield. The conditions are then changed to push the system out-of-spec (Frac-5, bottom panel), demonstrating the decrease of the product and increase of a reagent and side product.

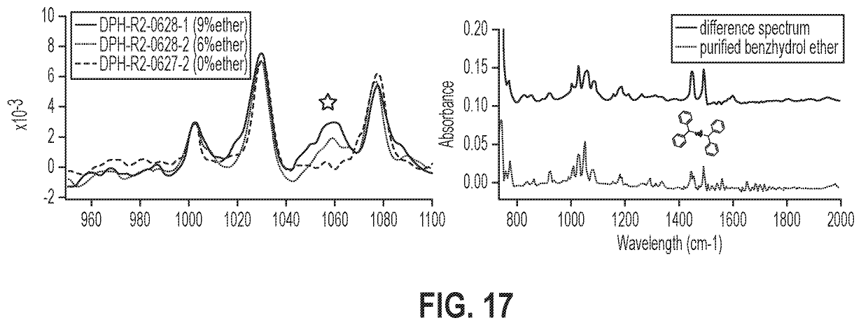

The results of chlorination are shown in Table 3. The FTIR was used off-line to determine the products and results are shown in FIG. 17. It is understood that the chlorination step is a key reaction that should be monitored. It was found that the FTIR was a more useful analytical tool in the chlorination step than Mass Spectrometer (MS) due to poor differentiation of the principal components by MS (Diphenylmethanol, chlorodiphenylmethane, and benzhydryl ether all fragment readily to diphenylmethyl cation). A similar ‘in-spec’/‘out-of-spec’ experiment was performed using a simplified configuration and FTIR data collected off-line.

TABLE 3
Chlorination

| Fraction | Composition (¹H NMR) | Note |
|---|---|---|
| 1 | 91% —Cl (chlorodiphenylmethane); 15% | Out of spec |
| | ether | |
| 2 | 94% —Cl; 6% ether | Equil. |
| 3 | 97% —Cl; 3% ether | Optimal |

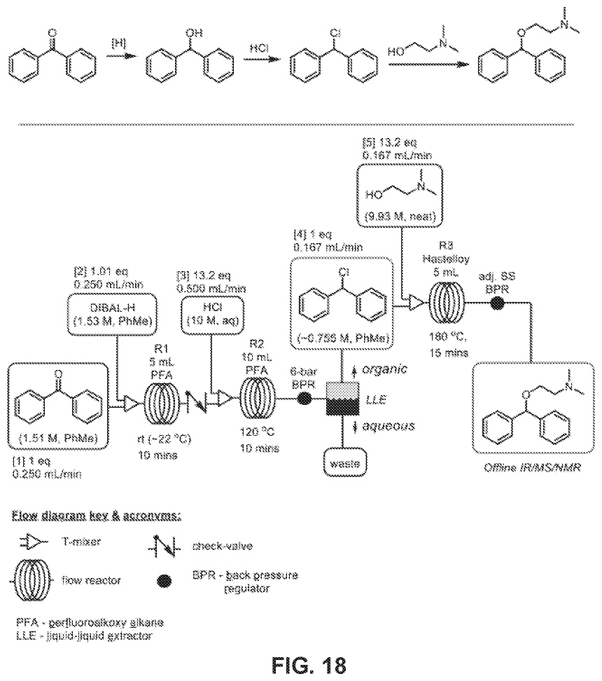

The schematics of the 3-Step process of using the disclosed system are shown in FIG. 18.

TABLE 4
3-Step Process

| Fraction | Composition (¹H NMR) | Note |
|---|---|---|
| 1 | 63% DPH (diphenylhydramine); 7% DPM | Equil. |
| | (diphenylmethanol), 21% —Cl, trace ether | |
| 2 | 82% DPH; 7% DPM; trace —Cl; trace ether | Equil. |
| 3 | 83% DPH; 7% DPM; trace —CL; trace ether | Optimal |
| 4 | 83% DPH; 6% DPM; trace —CL; trace ether | Optimal |
| 5 | 75% DPH; 14% DPM; trace —CL; trace ether | Shutdown |
| | | reaction |
| 6 | 56% DPH; 31% DPM; trace —CL; trace ether | Shutdown |
| | | reaction |

### Example 12

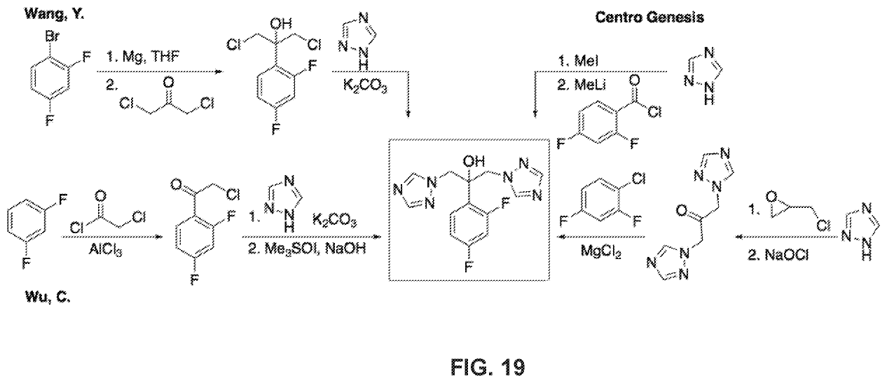

In this example an anti-fungal fluconazole was formed using an exemplary system as disclosed herein. As one of ordinary skill in the art would readily appreciate, the current synthesis of fluconazole involves only batch chemistry (FIG. 19) (for example, according to Wang, Assoc. J. Chem., 2014 26(24), 8593; or Wu, Zhongguo Yawu Huaxue Zazhil, 2011, 21(4), 304; or Jinana Luofeng Pharmaceutical Technology Co.). A continuous, three-step synthesis of fluconazole from 2-chloro-2′,4′-difluoroacetophenone was achieved with no need in intermediate purification.

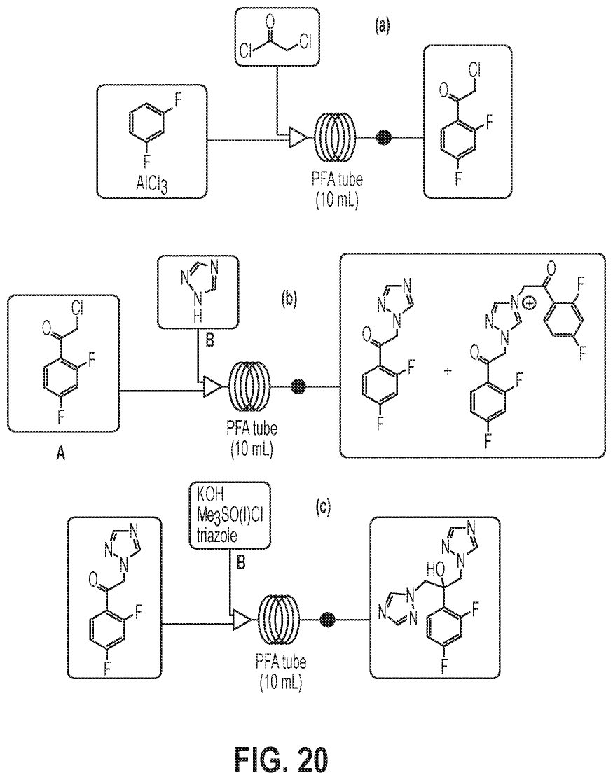

Fluconazole is a first-generation bis-triazole antifungal medicine commonly utilized to treat invasive infections caused by Candidas. Single step reactions were optimized using a commercially available flow system (FIG. 20). The reaction comprised a Friedel-Crafts Reaction (FIG. 20 (a)), Aklylation reaction (FIG. 20 (b)) and Epoxidation/Opening reaction (FIG. 20 (c)). Friedel-Crafts reaction was performed by flowing a solution of difluorobenzene (1.0 equiv, 8.7 M) and AlCl₃ (1.05 equiv, 4.9 M) in NO₂Me to react with neat chloroacetyl chloride (1.05 equiv, 12.5 M) unless specified. The results are shown in Table 5.

TABLE 5
Friedel-Crafts Reaction.

| Entry | Time (min) | Temp. (° C.) | Conversion (%)^{a} |
|---|---|---|---|
| 1 | 5 | 50 | 0 |
| 2 | 5 | 60 | 22 |
| 3 | 10 | 60 | 45 |
| 4 | 15 | 60 | 46 |
| 5 | 15 | 80 | 69^{b} |
| 6 | 15 | 90 | 77^{b} |
| 7^{c} | 25 | 70 | 74 |
| 8^{c} | 25 | 80 | 77 |
| 9^{d} | 25 | 80 | 79 |

^{a}Percent conversion was determine by crude ¹H NMR after working up the reaction.
^{b}The crude NMR contains the product and aromatic impurities.
^{c}Chloroacetyl chloride (1.15 equiv) and AlCl₃ (1.15 equiv).
^{d}Chloroacetyl chloride (1.3 equiv) and AlCl₃ (1.15 equiv).

Alkylation reaction was performed by flowing a solution of 2-chloro-2′,4′-difluoroacetophenone with a solution of triazole. The results are shown in Table 6.

TABLE 6
Alkylation reaction

| | | | | | Conver- |
|---|---|---|---|---|---|
| | | | Time | Temp. | sion |
| Entry | [A] | [B] | (min) | (° C.) | (%)^{a} |
| 1 | 1M toluene | 2.5M toluene:H₂O(1:1) | 15 | 150 | 13 |
| 2 | 1M IPA | 2.5M IPA:H₂O(1:1) | 15 | 150 | 26 |
| 3 | 1M dioxane | 2.5M dioxane:H₂O(1:1) | 15 | 150 | 33 |
| 4 | 1M MeCN | 2.5M MeCN:H₂O(1:1) | 15 | 150 | 43 |
| 5 | 1M NMP | 2.5M NMP:H₂O(1:1) | 15 | 150 | 71 |
| 6 | 1M DMSO | 2.5M DMSO:H₂O(1:1) | 15 | 150 | 60 |
| 7 | 1M NMP | 2.5M DMSO:H₂O(1:1) | 35 | 150 | 93 |
| 8 | 1M DMSO | 2.5M NMP:H₂O(1:1) | 35 | 150 | 74 |
| 9 | 0.2M DMSO | 1M DMSO:H₂O(1:1) | 45 | 150 | 99 |
| 10 | 0.2M NMP | 1M MP:H₂O(1:1) | 45 | 150 | 95 |
| 11 | 1M NMP | 10M NMP:H₂O(1:1)^{b} | 35 | 130 | 70^{c} |
| 12 | 1M NMP | 15M NMP:H₂O(1:1)^{b} | 35 | 130 | 82^{c} |
| 13 | 1M NMP | 20M NMP:H₂O(1:2)^{b} | 35 | 130 | 83^{c} |

^{a}Percent conversion was determined by crude ¹H NMR after working up the reaction unless specified. Over alkylated side-product was not present after work up.
^{b}As the concentration of triazole increased the over alkylated side-product decrease.
^{c}Percent conversion was determined by LCMS of the crude mixture.

Epoxidation/opening reaction was performed by flowing a solution of triazole acetophenone intermediate with a solution of KOH, Me₃SOCl, and triazole unless specified. The results are shown in Table 7.

TABLE 7
Epoxidation/Opening Reaction.

| | | | | Time | T | Convers. |
|---|---|---|---|---|---|---|
| Entry | [A] | [B] | [B] | (min) | (° C.) | (%)^{a} |
| 1 | 0.1M | 0.11M | 15% H₂O:DMSO | 35 | 80 | 57 |
| 2 | 0.1M | 0.11M | 15% H₂O:DMSO | 35 | 100 | 80 |
| 3 | 0.1M | 0.11M | 15% H₂O:DMSO | 35 | 150 | 71 |
| 4 | 0.18M | 0.22M | NMP:DMSO:H₂O(0.5:0.5:1) | 20 | 150 | 87 |
| 5 | 0.18M | 0.22M | NMP:DMSO:H₂O(0.5:0.5:1) | 20 | 120 | 79 |
| 6 | 0.18M | 0.22M | NMP:DMSO:H₂O(0.5:0.5:1) | 45 | 110 | 91 |
| 7^{b} | 0.25M | 0.29M | DMSO:H₂O(1:1) | 40 | 100 | 35^{c} |
| 8^{b} | 0.25M | 0.29M | NMP:H₂O(1:1) | 40 | 100 | 43^{c} |
| 9^{b} | 0.6M | 0.72M | DMSO:H₂O(1:1) | 40 | 100 | 60^{c} |
| 10^{b} | 0.6M | 0.72M | NMP:H₂O(1:1) | 40 | 100 | 69^{c} |

^{a}Percent conversion was determined by crude ¹H NMR after working up the reaction unless specified.
^{b}The reaction was performed using Me₃SOCl.
^{c}Percent conversion was determined by LCMS of the crude mixture.

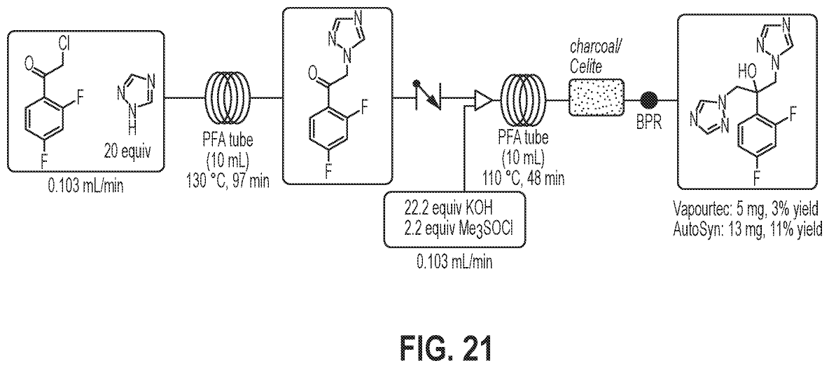

It was shown that these processes translated smoothly to the system components described above. A multistep synthesis was developed to afford fluconazole continuously and in high purity. FIG. 21 shows a schematic of three-step synthesis in the disclosed system.

It was shown that fluconazole can be successfully synthesized in three-step synthesis by flow chemistry on the disclosed modular reaction system. The synthesis of fluconazole begins with an alkylation of a solution of 2-chloro-2′,4′-difluoroacetophenone and triazole (20 equiv) to produce triazole acetophenone intermediate. Then, the triazole intermediate continues to react with a solution of KOH (22.2 equiv) and Me₃SOCl (2.2 equiv) to form the epoxide intermediate follow by epoxide opening with excess triazole to give fluconazole as the final product. The crude reaction can be purified by celite:charcoal column at the end of the reaction in high purity. It is believed that a four-step synthesis of fluconazole that includes the Friedel-Crafts reaction can be also developed using the inventive platform.

### Example 13

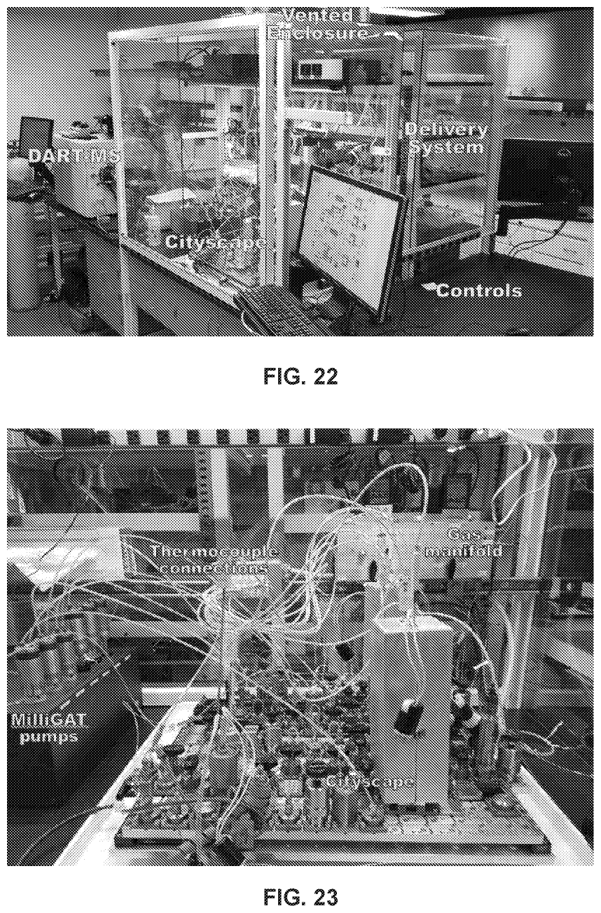

An exemplary automated synthesis platform as disclosed herein is shown in FIG. 22 was utilized. FIG. 22 shows a photograph of the overall view of the system that can be utilized in various syntheses. FIG. 23 shows a close up photograph of ventilated polycarbonate enclosure for a reaction platform as disclosed herein. The system can allow automated synthesis of at least one target from the start-up to the shut-down, while also providing the ability to switch between at least 2 targets in less than 2 hours using various valves to select the flow-path, as shown in FIG. 24, while allowing in-line and off-line characterization of the process steps and formed products.

Specifically, FIG. 24 shows exemplary synthetic routes for exemplary compounds such as: tranexamic acid, diazepam, nevirapine, warfarin, fluconazole, and diphenhydramine. It can be seen that a number of pathways can be about 511 possible pathways (without accounting for parallel reactors) or about 3,887 possible pathways (if parallel reactors and their resulting different residence times are taken into account).

FIG. 25 shows a photograph (FIG. 25 (a)) and a schematic (FIG. 25 (b)) of an exemplary platform configuration as disclosed herein. FIG. 26 shows an exemplary schematic of an integrated user interface.

FIG. 27 shows an exemplary pathway for the synthesis of diphenhydramine in 3 steps with 61% conversion. A solution of diphenylmethanol (0.8 M in toluene) was flowed through a reactor at 120° C. and contacted with hydrochloric acid (6 M aqueous) to generate a mixed stream containing diphenylchloride and aqueous waste. This stream passed through a liquid-liquid separator to separate an organic layer, which was then reacted with aminoethanol in a reactor at 180° C. to generate diphenhydramine at 61% conversion.

FIG. 28 shows an exemplary pathway for the synthesis of fluconazole in 3 steps with 78% conversion. A solution of acetophenone was reacted with triazole (20 eq.) at 130° C. This stream was then reacted with potassium hydroxide and trimethylsulfoniumiodide to produce a mixture containing fluconazole. This mixture then passed over an in-line charcoal filter to generate a stream containing fluconazole at 78% purity.

FIG. 29 shows an exemplary pathway for the synthesis of tranexamic acid in 1 step with 57% conversion. A hydrogenation was performed by flowing 4-aminomethyle-benzoic acid over a packed bed of platinum oxide at 75° C. and contacting with hydrogen to generate tranexamic acid at 57% conversion.

FIG. 30 shows an exemplary pathway for the synthesis of hydroxychloroquine in 1 step with 25% conversion. A solution of dichloroquinoline was reacted with aminoalcohol at 180° C. to produce a stream of hydroxychloroquine at 25% purity.

FIG. 31 shows an exemplary pathway for the synthesis of diazepam in 4 steps with 65% conversion. A solution of amino-benzophenone was mixed with an acid chloride in a reactor at room temperature, and then reacted with ammonium acetate and hexamethylenetetramine at 120° C. The resulting mixture was reacted with a solution of sodium methoxide to generate nordiazepam. The stream of nordiazepam was reacted with dimethylsulfate at 75° C. to produce diazepam at 65% purity.

FIG. 32 shows an exemplary pathway for the synthesis of (S)-warfarin in 1 step with 52% conversion and an enantiomeric excess of 89%. A solution of (E)-4-phenyl-3-buten-2-one was reacted with 4-hydroxycoumar in at 50° C. to generate a stream of (S)-(−) warfarin at 52% purity with an enantiomeric excess of 89%.

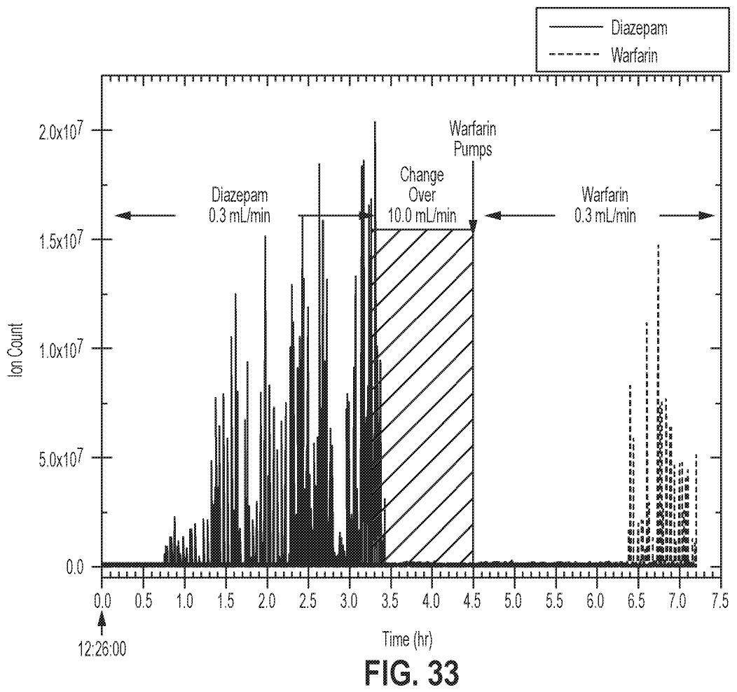

FIG. 33 shows ion counts as a function of time when the synthesis is switched from Diazepam to Warfarin in 1.2 hours. Thus, with the disclosed system, the user can easily switch from synthesis of one compound to synthesis of another compound within a matter of about an hour. This time window includes flushing the system of any by-products from the previous reaction and initializing and setting up the subsequent reaction to run.

Other reactions performed utilizing an inventive system include but are not limited to synthesis of diazepam, warfarin and the like.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1. A modular chemical reaction system comprising: a substrate layer having a substrate and a plurality of flow components positioned within the substrate, the substrate having an outer surface; a plurality of modules selectively mounted to the outer surface of the substrate in overlying relation to the plurality of flow components, wherein the plurality of modules cooperate with the plurality of flow components to form a fluid flow pathway for performing at least one step of a chemical reaction, the plurality of modules comprising: at least one process module, each process module of the plurality of process modules corresponding to a location of a step of the chemical reaction; and at least one regulator module, each regulator module of the plurality of regulator modules being positioned in fluid or thermal communication with the fluid flow pathway and configured to achieve, maintain, and/or measure one or more desired conditions of the chemical reaction; and at least one analysis device, each analysis device being positioned in operative communication with the fluid flow pathway through at least one module and configured to produce at least one output indicative of at least one characteristic of the chemical reaction as the chemical reaction occurs; and processing circuitry communicatively coupled to the plurality of modules and the at least one analysis device, wherein the processing circuitry is configured to receive the at least one output from the at least one analysis device and to use the at least one output to adjust operation of the at least one process module and the at least one regulator module to optimize the chemical reaction.

Aspect 2. The system of aspect 1, wherein the at least one process module comprises a reactor or a separator.

Aspect 3. The system of aspect 2, wherein the at least one process module comprises a reactor, and wherein the reactor is a vertical flow reactor, a heated tube reactor, or a reactor bed.

Aspect 4. The system of aspect 2 or aspect 3, wherein the at least one process module comprises a separator, and wherein the separator is a liquid/liquid separator or a liquid/gas separator.

Aspect 5. The system of any one of the preceding aspects, wherein the plurality of flow components comprise a plurality of flow connectors, wherein each flow connector is configured to selectively: form a portion of the fluid flow pathway for performing the chemical reaction; or be disengaged from flow connectors forming the fluid flow pathway such that the flow connector is not in fluid communication with the fluid flow pathway.

Aspect 6. The system of any one of the preceding aspects, wherein the at least one regulator module comprises: a check valve, a tee filter, a flow regulator, a pressure sensing module, a pressure relief valve, a pressure regulator, a tube adaptor, a valve, a pump, a flow stream selector, a control valve module, a temperature monitoring module, a temperature control module, a heater, or a cooler.

Aspect 7. The system of any one of the preceding aspects, wherein the analysis device comprises: a UV-Vis spectrometer, a near-infrared (NIR) spectrometer, a Raman spectrometer, a Fourier Transform-Infrared (FT-IR) spectrometer, a nuclear magnetic resonance (NMR) spectrometer, or a mass spectrometer (MS).

Aspect 8. The system of any one of preceding aspects, wherein the fluid flow pathway is a liquid flow pathway.

Aspect 9. A modular chemical reaction system comprising: a substrate layer having a substrate and a plurality of flow components positioned within the substrate, the substrate having an outer surface; a surface-mount layer having a plurality of flow modules selectively mounted to the outer surface of the substrate in overlying relation to the plurality of flow components, wherein each flow module of the plurality of flow modules is positioned in fluid communication with at least one flow component of the plurality of flow components at a respective interface; and a plurality of sealing elements configured to establish a fluid-tight seal at each interface between a flow module of the plurality of flow modules and a flow component of the plurality of flow components, wherein the plurality of flow modules and the plurality of flow components cooperate to establish a fluid flow pathway for performing at least one step of a chemical reaction, and wherein at least one flow module of the plurality of flow modules is a reactor or a separator.

Aspect 10. The modular chemical reaction system of aspect 9, further comprising at least one regulator module selectively mounted to the outer surface of the substrate, wherein each regulator module of the at least one regulator module is configured to achieve, maintain, and/or modify one or more desired conditions of the chemical reaction.

Aspect 11. The modular chemical reaction system of aspect 10, further comprising at least one analysis device, each analysis device of the at least one analysis device being positioned in operative communication with the fluid flow pathway and configured to produce at least one output indicative of at least one characteristic of the chemical reaction as the chemical reaction occurs.

Aspect 12. The modular chemical reaction system of aspect 11, wherein a first flow module of the plurality of flow modules defines an analysis outlet that is configured for positioning in operative communication with the analysis device.

Aspect 13. The modular chemical reaction system of aspect 12, wherein the first flow module is positioned upstream of at least one other flow module of the plurality of flow modules.

Aspect 14. The modular chemical reaction system of aspect 11 or aspect 12, further comprising processing circuitry communicatively coupled to the at least one analysis device and at least a portion of the plurality of flow modules, wherein the processing circuitry is configured to receive the at least one output from the at least one analysis device and to use the at least one output to adjust operation of at least one flow module of the plurality of flow modules to optimize the chemical reaction.

Aspect 15. The modular chemical reaction system of any one of aspects 9-14, further comprising a manifold layer comprising at least one manifold body underlying the substrate layer, wherein the plurality of flow connectors comprises a first plurality of flow connectors positioned within the substrate layer and a second plurality of flow connectors positioned within the manifold layer.

Aspect 16. The modular chemical reaction system of any one of aspects 9-15, wherein each flow connector of the plurality of flow connectors has an inner diameter ranging from about 0.04 inches to about 0.08 inches.

Aspect 17. The modular chemical reaction system of any one of aspects claims 9-16, wherein the at least one flow module that is a reactor or a separator has a fluid inlet portion and a fluid outlet portion, wherein at least one of the fluid inlet portion and the fluid outlet portion of the at least one flow module shares a consistent inner diameter with an adjacent flow connector of the plurality of flow connectors.

Aspect 18. The modular chemical reaction system of any one of aspects 9-17, wherein the fluid flow pathway is a liquid flow pathway, and wherein the plurality of sealing elements are configured to establish a liquid-tight seal at each interface between a flow module of the plurality of flow modules and a flow component of the plurality of flow components.

Aspect 19. The modular chemical reaction system of any one of aspects 9-18, wherein at least one flow module of the plurality of flow modules comprises a reactor.

Aspect 20. The modular chemical reaction system of aspect 19, wherein the reactor is a heated tube reactor.

Aspect 21. The modular chemical reaction system of any one of aspects 9-20, wherein at least one flow module of the plurality of flow modules comprise a separator.

Aspect 22. The modular chemical reaction system of aspect 21, wherein the separator is a liquid-liquid separator.

Aspect 23. The modular chemical reaction system of aspect 22, wherein the separator is a membrane-based liquid-liquid separator.

Aspect 24. The modular chemical reaction system of claim 22, wherein the separator is a gravity-based liquid-liquid separator.

Aspect 25. The modular chemical reaction system of aspect 21, wherein the separator is a gas-liquid separator.

Aspect 26. The modular chemical reaction system of aspect 25, wherein the separator is a gravity-based gas-liquid separator.

Aspect 27. The modular chemical reaction system of any one of aspects 9-26, further comprising: at least one sensor positioned in fluid communication with a first flow module of the plurality of flow modules, wherein each sensor of the at least one sensor is configured for producing an output indicative of at least one characteristic of liquid within the first flow module; and processing circuitry communicatively coupled to the at least one sensor.

Aspect 28. A reactor comprising: a body defining an interior chamber and an inlet and an outlet in fluid communication with the interior chamber, wherein the body of the reactor is selectively mountable to an upper surface of a substrate layer to respectively establish fluid communication between the inlet and outlet of the body and respective portions of a fluid flow pathway at least partially defined within the substrate layer.

Aspect 29. A separator comprising: a body defining an interior chamber and an inlet and an outlet in fluid communication with the interior chamber, wherein the body of the separator is selectively mountable to an upper surface of a substrate layer to respectively establish fluid communication between the inlet and outlet of the body and respective portions of a liquid flow pathway at least partially defined within the substrate layer.

Aspect 30. An analytical flow cell comprising: a body defining an interior chamber and an analysis outlet in fluid communication with the interior chamber, wherein the body of the flow cell is selectively mountable to an upper surface of a substrate layer to respectively establish fluid communication between the first inlet and the first outlet of the body and respective portions of a liquid flow pathway at least defined within the substrate layer, and wherein the analysis outlet of the body is configured for positioning in fluid communication with a analysis device.

Aspect 31. A method comprising: introducing at least one liquid reagent into the fluid flow pathway of the system of any one of claims 1-8; and performing at least one step of a chemical reaction using the at least one liquid reagent.

Aspect 32. The method of aspect 31, wherein the at least one process module comprises a plurality of process modules, and wherein the chemical reaction is a multi-step chemical synthesis comprising a plurality of sequential steps, each step of the plurality of sequential steps corresponding to flow of reagents within a respective process module.

Aspect 33. The method of aspect 31 or aspect 32, further comprising: mounting an additional process module to the outer surface of the substrate, wherein the additional process module is a reactor or a separator; establishing fluid communication between the additional process module and the fluid flow pathway; and running at least one step of a second chemical reaction using a modified fluid flow pathway including the additional process module.

Aspect 34. The method of any one of aspects 31-33, further comprising: using the processing circuitry to receive the at least one output from the at least one analysis device; and using the process circuitry to adjust operation of the at least one process module and the at least one regulator module to optimize the chemical reaction.

Aspect 35. A method comprising: introducing at least one liquid reagent into the fluid flow pathway of the system of any one of aspects 9-30; and performing at least one step of a chemical reaction using the at least one liquid reagent.

Aspect 36. The method of aspect 36, wherein the chemical reaction is a multi-step chemical synthesis comprising a plurality of sequential steps, each step of the plurality of sequential steps corresponding to flow of reagents within at least one flow module of the plurality of flow modules.

Aspect 37. The method of aspect 35 or aspect 36, further comprising modifying the liquid flow pathway without disconnecting any flow modules of the plurality of flow modules from the substrate layer or adjusting a position of any flow connectors of the plurality of flow connectors relative to the plurality of flow modules.

Aspect 38. The method of aspect 37, wherein at least one flow module of the plurality of flow modules comprises a flow valve that is selectively adjustable among at least first and second flow positions that are configured to produce different flow characteristics through the flow valve, and wherein modifying the liquid flow pathway comprises selectively moving the flow valve about and between at least the first and second flow positions.

Aspect 39. The method of any one of aspects 35-38, further comprising: mounting an additional flow module of the plurality of flow modules to the outer surface of the substrate layer, wherein the additional flow module is a reactor or a separator; and establishing fluid communication between the additional flow module and the liquid flow pathway.

Aspect 40. A modular chemical reaction system comprising: a substrate layer having a substrate and a plurality of flow components positioned within the substrate, the substrate having an outer surface; a plurality of modules selectively mounted to the outer surface of the substrate in overlying relation to the plurality of flow components, wherein the plurality of modules cooperate with the plurality of flow components to produce a first configuration that forms a first fluid flow pathway for performing at least one step of a first chemical reaction, the plurality of modules comprising at least one monitoring module configured to produce at least one output indicative of at least one condition of the first chemical reaction; at least one analysis device, each analysis device being positioned in operative communication with the fluid flow pathway through at least one module of the plurality of modules and configured to produce at least one output indicative of at least one characteristic of the chemical reaction as the chemical reaction occurs; and processing circuitry communicatively coupled to the at least one monitoring module and the at least one analysis device, wherein the processing circuitry is configured to receive the outputs from the at least one monitoring module and the at least one analysis device to monitor the chemical reaction as the chemical reaction occurs, and wherein the plurality of modules and the flow components within the substrate layer are configured for selective rearrangement to a second configuration within a minimal changeover period to produce a second fluid flow pathway for performing at least one step of a second chemical reaction.

Aspect 41. The modular chemical reaction system of aspect 40, wherein the processing circuitry comprises at least one control module that is selectively mountable to the outer surface of the substrate.

Aspect 42. The modular chemical reaction system of aspect 40, wherein the plurality of modules comprises at least one process module corresponding to a location of a step of the chemical reaction, and wherein the plurality of monitoring modules comprises at least one regulator module, each regulator module being positioned in fluid or thermal communication with the fluid flow pathway and configured to achieve, maintain, and/or measure one or more desired conditions of the chemical reaction.

Aspect 43. The modular chemical reaction system of aspect 42, wherein the processing circuitry is configured to use the outputs from the at least one monitoring module and the at least one analysis device to adjust operation of the at least one process module and the at least one regulator module to optimize the chemical reaction.

Aspect 44. The system of aspect 42 or aspect 43, wherein the at least one process module comprises a reactor or a separator.

Aspect 45. The system of aspect 44, wherein the at least one process module comprises a reactor, and wherein the reactor is a heated tube reactor or a packed bed reactor.

Aspect 46. The system of claim aspect 44, wherein the at least one process module comprises a separator, and wherein the separator is a liquid/liquid separator or a liquid/gas separator.

Aspect 47. The system of any one of aspects 40-46, wherein the plurality of flow components comprise a plurality of flow connectors, wherein each flow connector is configured to selectively: form a portion of the fluid flow pathway for performing the chemical reaction; or be disengaged from flow connectors forming the fluid flow pathway such that the flow connector is not in fluid communication with the fluid flow pathway.

Aspect 48. The system of aspect 42 or aspect 43, wherein the at least one regulator module comprises a plurality of regulator modules, wherein the first and second configurations of the plurality of modules and the plurality of flow components comprise respective first and second arrangements of regulator modules, wherein the first and second arrangements of regulator modules differ from one another and comprise at least five of the following: a check valve, a tee filter, a flow regulator, a pressure sensing module, a pressure relief valve, a pressure regulator, a tube adaptor, a valve, a pump, a control valve module, a temperature monitoring module, a temperature control module, a heater, or a cooler.

Aspect 49. The system of aspect 42 or aspect 43, wherein the at least one analysis device comprises a plurality of analysis devices, wherein a first configuration of the plurality of analysis devices is in operative communication with the first fluid flow pathway, wherein the plurality of modules and the flow components within the substrate layer are configured for selective rearrangement to establish operative communication between a second configuration of the plurality of analysis devices and the second fluid flow pathway, and wherein the first and second configurations of the plurality of analysis devices include at least two of the following: a UV-Vis spectrometer, a near-infrared (NIR) spectrometer, a Raman spectrometer, a Fourier Transform-Infrared (FT-IR) spectrometer, a nuclear magnetic resonance (NMR) spectrometer, or a mass spectrometer (MS).

Aspect 50. The system of any one of aspects 40-49, wherein the fluid flow pathway is a liquid flow pathway.

Aspect 51. A method comprising: introducing at least one reagent into the fluid flow pathway of the system of any one of aspects 40-50; and performing at least one step of a chemical reaction using the at least one reagent.

Aspect 52. The method of aspect 51, wherein the at least one module comprises a plurality of process modules, and wherein the chemical reaction is a multi-step chemical synthesis comprising a plurality of sequential steps, each step of the plurality of sequential steps corresponding to flow of reagents within a respective process module.

Aspect 53. The method of aspect 52, further comprising: mounting an additional process module to the outer surface of the substrate, wherein the additional process module is a reactor or a separator; establishing fluid communication between the additional process module and the fluid flow pathway; and running at least one step of a second chemical reaction using a modified fluid flow pathway including the additional process module.

Aspect 54. The method of aspect 52, further comprising: using the processing circuitry to receive the outputs from the at least one monitoring module and at least one analysis device; and using the process circuitry to adjust operation of the at least one process module to optimize the chemical reaction. Aspect 55. A modular chemical reaction system comprising: a substrate layer having a substrate and a plurality of flow components positioned within the substrate, the substrate having an outer surface; a plurality of modules selectively mounted to the outer surface of the substrate in overlying relation to the plurality of flow components, wherein at least a portion of the plurality of modules cooperate with at least a portion of the plurality of flow components to produce a first fluid flow pathway for performing at least one step of a first chemical reaction, the plurality of modules comprising at least one monitoring module configured to produce at least one output indicative of at least one condition of the first chemical reaction; at least one analysis device, each analysis device being positioned in operative communication with the fluid flow pathway through at least one module of the plurality of modules and configured to produce at least one output indicative of at least one characteristic of the chemical reaction as the chemical reaction occurs; and processing circuitry communicatively coupled to the at least one monitoring module and the at least one analysis device, wherein the processing circuitry is configured to receive the outputs from the at least one monitoring module and the at least one analysis device to monitor the chemical reaction as the chemical reaction occurs, and wherein the plurality of modules and the flow components within the substrate layer are configured for selective rearrangement within a minimal changeover period to produce a second fluid flow pathway for performing at least one step of a second chemical reaction, the second fluid flow pathway being different than the first fluid flow pathway.

Aspect 56. The modular chemical reaction system of aspect 55, wherein the processing circuitry comprises at least one control module that is selectively mountable to the outer surface of the substrate.

Aspect 57. The modular chemical reaction system of aspect 55 or aspect 56, wherein the plurality of modules comprises at least one process module corresponding to a location of a step of the chemical reaction, and wherein the plurality of monitoring modules comprises at least one regulator module, each regulator module being positioned in fluid or thermal communication with the fluid flow pathway and configured to achieve, maintain, and/or measure one or more desired conditions of the chemical reaction.

Aspect 58. The modular chemical reaction system of aspect 57, wherein the processing circuitry is configured to use the outputs from the at least one monitoring module and the at least one analysis device to adjust operation of the at least one process module and the at least one regulator module to optimize the chemical reaction.

Aspect 59. The system of aspect 57 or aspect 58, wherein the at least one process module comprises a reactor or a separator.

Aspect 60. The system of aspect 59, wherein the at least one process module comprises a reactor, and wherein the reactor is a heated tube reactor or a packed bed reactor.

Aspect 61. The system of aspect 59, wherein the at least one process module comprises a separator, and wherein the separator is a liquid/liquid separator or a liquid/gas separator.

Aspect 62. The system of any one of aspects 55-61, wherein the plurality of flow components comprise a plurality of flow connectors, wherein each flow connector is configured to selectively: form a portion of the fluid flow pathway for performing the chemical reaction; or be disengaged from flow connectors forming the fluid flow pathway such that the flow connector is not in fluid communication with the fluid flow pathway.

Aspect 63. The system of aspect 57 or aspect 58, wherein the at least one regulator module comprises a plurality of regulator modules, wherein the first and second fluid flow pathways are at least partially defined by respective first and second arrangements of regulator modules, wherein the first and second arrangements of regulator modules differ from one another and comprise at least five of the following: a check valve, a tee filter, a flow regulator, a pressure sensing module, a pressure relief valve, a pressure regulator, a tube adaptor, a valve, a pump, a control valve module, a temperature monitoring module, a temperature control module, a heater, or a cooler.

Aspect 64. The system of aspect 57 or aspect 58, wherein the at least one analysis device comprises a plurality of analysis devices, wherein a first configuration of the plurality of analysis devices is in operative communication with the first fluid flow pathway, wherein the plurality of modules and the flow components within the substrate layer are configured for selective rearrangement to establish operative communication between a second configuration of the plurality of analysis devices and the second fluid flow pathway, and wherein the first and second configurations of the plurality of analysis devices include at least two of the following: a UV-Vis spectrometer, a near-infrared (NIR) spectrometer, a Raman spectrometer, a Fourier Transform-Infrared (FT-IR) spectrometer, a nuclear magnetic resonance (NMR) spectrometer, or a mass spectrometer (MS).

Aspect 65. The system of any one of aspects 55-64, wherein the fluid flow pathway is a liquid flow pathway.

Aspect 66. The system of any one of aspects 55-65, wherein the plurality of modules and the plurality of flow connectors are configured to permit modification of the first fluid flow pathway to the second fluid flow pathway without changing locations of the plurality of modules and the plurality of flow connectors with respect to the substrate, and wherein the second fluid flow pathway comprises at least one module that did not define a portion of the first fluid flow pathway.

Aspect 67. A method comprising: introducing at least one reagent into the first fluid flow pathway of the system of any one of aspects 55-66; and performing at least one step of a chemical reaction using the at least one reagent.

Aspect 68. The method of aspect 67, further comprising: modifying the first fluid flow pathway using the plurality of modules and the plurality of flow components; and running at least one step of a second chemical reaction using the modified fluid flow pathway, wherein the plurality of modules and the flow components within the substrate layer are selectively rearranged to produce the modified fluid flow pathway within a minimal changeover period.

Aspect 69. The method of aspect 68, wherein locations of the plurality of modules and the flow components within the substrate layer are not changed with respect to the substrate, and wherein the modified fluid flow pathway comprises at least one module that did not define a portion of the first fluid flow pathway.

Aspect 70. The method of any one of aspects 67-69, further comprising: using the processing circuitry to receive the outputs from the at least one monitoring module and at least one analysis device; and using the process circuitry to adjust operation of the at least one process module to optimize the chemical reaction.

Aspect 71. A modular chemical reaction system comprising: a substrate layer having a substrate and a plurality of flow components positioned within the substrate, the substrate having an outer surface; a plurality of modules selectively mounted to the outer surface of the substrate in overlying relation to the plurality of flow components, wherein at least a portion of the plurality of modules cooperate with at least a portion of the plurality of flow components to produce a first fluid flow pathway for performing at least one step of a first chemical reaction, the plurality of modules comprising at least one monitoring module configured to produce at least one output indicative of at least one condition of the first chemical reaction; and processing circuitry communicatively coupled to the at least one monitoring module, wherein the processing circuitry is configured to receive the outputs from the at least one monitoring module to monitor the chemical reaction as the chemical reaction occurs, and wherein the plurality of modules and the flow components within the substrate layer are configured for selective rearrangement within a minimal changeover period to produce a second fluid flow pathway for performing at least one step of a second chemical reaction, the second fluid flow pathway being different than the first fluid flow pathway.

What is claimed is:

1. A modular chemical reaction system comprising:
a substrate layer having a substrate and a plurality of flow components positioned within the substrate, the substrate having an outer surface;
a plurality of modules selectively mounted to the outer surface of the substrate in overlying relation to the plurality of flow components, wherein at least a portion of the plurality of modules cooperate with at least a portion of the plurality of flow components to produce a first fluid flow pathway for performing at least one step of a first chemical reaction, the plurality of modules comprising at least one monitoring module configured to produce at least one output indicative of at least one condition of the first chemical reaction;
at least one analysis device, each analysis device being positioned in operative communication with the fluid flow pathway through at least one module of the plurality of modules and configured to produce at least one output indicative of at least one characteristic of the chemical reaction as the chemical reaction occurs; and
processing circuitry communicatively coupled to the at least one monitoring module and the at least one analysis device, wherein the processing circuitry is configured to receive the outputs from the at least one monitoring module and the at least one analysis device to monitor the chemical reaction as the chemical reaction occurs, and
wherein the plurality of modules and the flow components within the substrate layer are configured for selective rearrangement within a minimal changeover period to produce a second fluid flow pathway for performing at least one step of a second chemical reaction, the second fluid flow pathway being different than the first fluid flow pathway.

2. The modular chemical reaction system of claim 1, wherein the processing circuitry comprises at least one control module that is selectively mountable to the outer surface of the substrate.

3. The modular chemical reaction system of claim 1, wherein the plurality of modules comprises at least one process module corresponding to a location of a step of the chemical reaction, and wherein the plurality of monitoring modules comprises at least one regulator module, each regulator module being positioned in fluid or thermal communication with the fluid flow pathway and configured to achieve, maintain, and/or measure one or more desired conditions of the chemical reaction.

4. The modular chemical reaction system of claim 3, wherein the processing circuitry is configured to use the outputs from the at least one monitoring module and the at least one analysis device to adjust operation of the at least one process module and the at least one regulator module to optimize the chemical reaction.

5. The system of claim 3, wherein the at least one process module comprises a reactor or a separator.

6. The system of claim 5, wherein the at least one process module comprises a reactor, and wherein the reactor is a heated tube reactor or a packed bed reactor.

7. The system of claim 5, wherein the at least one process module comprises a separator, and wherein the separator is a liquid/liquid separator or a liquid/gas separator.

8. The system of claim 1, wherein the plurality of flow components comprise a plurality of flow connectors, wherein each flow connector is configured to selectively:
(a) form a portion of the fluid flow pathway for performing the chemical reaction; or
(b) be disengaged from flow connectors forming the fluid flow pathway such that the flow connector is not in fluid communication with the fluid flow pathway.

9. The system of claim 3, wherein the at least one regulator module comprises a plurality of regulator modules, wherein the first and second fluid flow pathways are at least partially defined by respective first and second arrangements of regulator modules, wherein the first and second arrangements of regulator modules differ from one another and comprise at least five of the following: a check valve, a tee filter, a flow regulator, a pressure sensing module, a pressure relief valve, a pressure regulator, a tube adaptor, a valve, a pump, a control valve module, a temperature monitoring module, a temperature control module, a heater, or a cooler.

10. The system of claim 3, wherein the at least one analysis device comprises a plurality of analysis devices, wherein a first configuration of the plurality of analysis devices is in operative communication with the first fluid flow pathway, wherein the plurality of modules and the flow components within the substrate layer are configured for selective rearrangement to establish operative communication between a second configuration of the plurality of analysis devices and the second fluid flow pathway, and wherein the first and second configurations of the plurality of analysis devices include at least two of the following: a UV-Vis spectrometer, a near-infrared (NIR) spectrometer, a Raman spectrometer, a Fourier Transform-Infrared (FT-IR) spectrometer, a nuclear magnetic resonance (NMR) spectrometer, or a mass spectrometer (MS).

11. (canceled)

12. The system of claim 1, wherein the plurality of modules and the plurality of flow connectors are configured to permit modification of the first fluid flow pathway to the second fluid flow pathway without changing locations of the plurality of modules and the plurality of flow connectors with respect to the substrate, and wherein the second fluid flow pathway comprises at least one module that did not define a portion of the first fluid flow pathway.

13. A modular chemical reaction system comprising:
a substrate layer having a substrate and a plurality of flow components positioned within the substrate, the substrate having an outer surface;
a surface-mount layer having a plurality of flow modules selectively mounted to the outer surface of the substrate in overlying relation to the plurality of flow components, wherein each flow module of the plurality of flow modules is positioned in fluid communication with at least one flow component of the plurality of flow components at a respective interface; and
a plurality of sealing elements configured to establish a fluid-tight seal at each interface between a flow module of the plurality of flow modules and a flow component of the plurality of flow components,
wherein the plurality of flow modules and the plurality of flow components cooperate to establish a fluid flow pathway for performing at least one step of a chemical reaction, and
wherein at least one flow module of the plurality of flow modules is a reactor or a separator.

14. The modular chemical reaction system of claim 13, further comprising at least one regulator module selectively mounted to the outer surface of the substrate, wherein each regulator module of the at least one regulator module is configured to achieve, maintain, and/or modify one or more desired conditions of the chemical reaction.

15. The modular chemical reaction system of claim 14, further comprising at least one analysis device, each analysis device of the at least one analysis device being positioned in operative communication with the fluid flow pathway and configured to produce at least one output indicative of at least one characteristic of the chemical reaction as the chemical reaction occurs.

16. The modular chemical reaction system of claim 15, wherein a first flow module of the plurality of flow modules defines an analysis outlet that is configured for positioning in operative communication with the analysis device.

17. (canceled)

18. The modular chemical reaction system of claim 15, further comprising processing circuitry communicatively coupled to the at least one analysis device and at least a portion of the plurality of flow modules, wherein the processing circuitry is configured to receive the at least one output from the at least one analysis device and to use the at least one output to adjust operation of at least one flow module of the plurality of flow modules to optimize the chemical reaction.

19. The modular chemical reaction system of claim 13, further comprising a manifold layer comprising at least one manifold body underlying the substrate layer, wherein the plurality of flow connectors comprises a first plurality of flow connectors positioned within the substrate layer and a second plurality of flow connectors positioned within the manifold layer.

20. The modular chemical reaction system of claim 13, wherein the at least one flow module that is a reactor or a separator has a fluid inlet portion and a fluid outlet portion, wherein at least one of the fluid inlet portion and the fluid outlet portion of the at least one flow module shares a consistent inner diameter with an adjacent flow connector of the plurality of flow connectors.

21. The modular chemical reaction system of claim 13, wherein the fluid flow pathway is a liquid flow pathway, and wherein the plurality of sealing elements are configured to establish a liquid-tight seal at each interface between a flow module of the plurality of flow modules and a flow component of the plurality of flow components.

22. A method comprising:
introducing at least one reagent into a first fluid flow pathway of a modular chemical reaction system, the modular chemical reaction system comprising:
a substrate layer having a substrate and a plurality of flow components positioned within the substrate, the substrate having an outer surface;
a plurality of modules selectively mounted to the outer surface of the substrate in overlying relation to the plurality of flow components, wherein at least a portion of the plurality of modules cooperate with at least a portion of the plurality of flow components to produce the first fluid flow pathway, the plurality of modules comprising at least one monitoring module configured to produce at least one output indicative of at least one condition of the first chemical reaction;
at least one analysis device, each analysis device being positioned in operative communication with the fluid flow pathway through at least one module of the plurality of modules and configured to produce at least one output indicative of at least one characteristic of the chemical reaction as the chemical reaction occurs; and
processing circuitry communicatively coupled to the at least one monitoring module and the at least one analysis device, wherein the processing circuitry is configured to receive the outputs from the at least one monitoring module and the at least one analysis device to monitor the chemical reaction as the chemical reaction occurs; and
performing at least one step of a chemical reaction using the at least one reagent.

23. The method of claim 22, further comprising:
modifying the first fluid flow pathway using the plurality of modules and the plurality of flow components; and
running at least one step of a second chemical reaction using the modified fluid flow pathway,
wherein the plurality of modules and the flow components within the substrate layer are selectively rearranged to produce the modified fluid flow pathway within a minimal changeover period.

24. (canceled)

25. (canceled)
